(19) United States
(12) Patent Application Publication
(10) Pub. No.: US 2014/0346197 A1
(43) Pub. Date: Nov. 27, 2014

(54) SYSTEMS AND METHODS FOR DELIVERING A THERAPEUTIC AGENT USING MECHANICAL ADVANTAGE

(71) Applicant: SpringLeaf Therapeutics, Inc., Boston, MA (US)

(72) Inventors: J. Richard Gyory, Sudbury, MA (US); Alessandro Pizzochero, Chelmsford, MA (US)

(73) Assignee: SpringLeaf Therapeutics, Inc., Boston, MA (US)

(21) Appl. No.: 14/164,949

(22) Filed: Jan. 27, 2014

Related U.S. Application Data
(63) Continuation of application No. 13/101,798, filed on May 5, 2011, now abandoned.
(60) Provisional application No. 61/331,766, filed on May 5, 2010.

(51) Int. Cl. A61M 5/142

(52) U.S. Cl. CPC A61M 5/142
USPC 222/372

(57) ABSTRACT

Devices and methods for delivering a therapeutic agent to a patient are disclosed herein. In one embodiment, a delivery system includes a reservoir configured to contain a fluid, a first actuator coupled to the reservoir, a second actuator coupled to the first actuator, and an adaptor at least partially disposed between the first actuator and the second actuator. The first actuator and the second actuator are configured to collectively exert a force on the reservoir such that at least a portion of the fluid within the reservoir is communicated out of the reservoir. The adaptor is configured to couple the first actuator and the second actuator.

### CROSS-REFERENCE TO RELATED APPLICATION

This application claims priority to and the benefit of U.S. Provisional Application Ser. No. 61/331,766, filed May 5, 2010, entitled “Systems and Methods for Delivering a Therapeutic Agent Using Mechanical Advantage,” the disclosure of which is hereby incorporated by reference herein in its entirety.

### BACKGROUND

The invention relates generally to medical devices and procedures, including, for example, medical devices and methods for delivering a therapeutic agent to a patient.

Drug delivery involves delivering a drug or other therapeutic compound into the body. Typically, the drug is delivered via a technology that is carefully selected based on a number of factors. These factors can include, but are not limited to, the characteristics of the drug, such as drug dose, pharmacokinetics, complexity, cost, and absorption, the characteristics of the desired drug delivery profile (such as uniform, non-uniform, or patient-controlled), the characteristics of the administration mode (such as the ease, cost, complexity, and effectiveness of the administration mode for the patient, physician, nurse, or other caregiver), or other factors or combinations of these factors.

Conventional drug delivery technologies present various challenges. Oral administration of a dosage form is a relatively simple delivery mode, but some drugs may not achieve the desired bioavailability and/or may cause undesirable side effects if administered orally. Further, the delay from time of administration to time of efficacy associated with oral delivery may be undesirable depending on the therapeutic need. While parenteral administration by injection may avoid some of the problems associated with oral administration, such as providing relatively quick delivery of the drug to the desired location, conventional injections may be inconvenient, difficult to self-administer, and painful or unpleasant for the patient. Furthermore, injection may not be suitable for achieving certain delivery/release profiles, particularly over a sustained period of time.

Passive transdermal technology, such as a conventional transdermal patch, may be relatively convenient for the user and may permit relatively uniform drug release over time. However, some drugs, such as highly charged or polar drugs, peptides, proteins and other large molecule active agents, may not penetrate the stratum corneum for effective delivery. Furthermore, a relatively long start-up time may be required before the drug takes effect. Thereafter, the drug release may be relatively continuous, which may be undesirable in some cases. Also, a substantial portion of the drug payload may be undeliverable and may remain in the patch once the patch is removed.

Active transdermal systems, including iontophoresis, sonophoresis, and poration technology, may be expensive and may yield unpredictable results. Only some drug formulations, such as aqueous stable compounds, may be suited for active transdermal delivery. Further, modulating or controlling the delivery of drugs using such systems may not be possible without using complex systems.

Some infusion pump systems may be large and may require tubing between the pump and the infusion set, which can impact the quality of life of the patient. Further, infusion pumps may be expensive and may not be disposable. From the above, it would be desirable to provide new and improved drug delivery systems and methods that overcome some or all of these and other drawbacks.

### SUMMARY OF THE INVENTION

Devices and methods for delivering a therapeutic agent to a patient are disclosed herein. In one embodiment, a delivery system includes a reservoir configured to contain a fluid, a first actuator coupled to the reservoir, a second actuator coupled to the first actuator, and an adaptor at least partially disposed between the first actuator and the second actuator. The first actuator and the second actuator are configured to collectively exert a force on the reservoir such that at least a portion of the fluid within the reservoir is communicated out of the reservoir. The adaptor is configured to couple the first actuator and the second actuator.

### BRIEF DESCRIPTION OF THE DRAWINGS

FIG. 1 is a schematic illustration of a delivery system according to an embodiment.

FIG. 2A is a side view of a schematic illustration of an electrochemical actuator shown in a charged state; and FIG. 2B is a schematic illustration of a side view of the electrochemical actuator of FIG. 2A shown in a discharged state.

FIG. 3A is a schematic illustration of a portion of a delivery system according to an embodiment illustrating an electrochemical actuator in a charged state and FIG. 3B is a schematic illustration of the portion of the delivery system of FIG. 3A illustrating the electrochemical actuator as it discharges.

FIG. 3C is a schematic illustration of a portion of a delivery system according to an embodiment illustrating an electrical circuit including a first electrochemical actuator and a second electrochemical actuator.

FIG. 4A is a perspective view of a delivery system according to an embodiment and FIG. 4B is an exploded view of the delivery system of FIG. 4A.

FIG. 5 is a side partial cross-sectional view of a delivery device according to an embodiment, shown in a first configuration.

FIG. 6 is a side partial cross-sectional view of the delivery device of FIG. 5, shown in a second configuration.

FIG. 7 is a schematic top view of the delivery device of FIG. 5 with a top portion of the housing removed.

FIG. 8 is a perspective view of a portion of the delivery device of FIG. 5, including the adaptor plate and electrochemical actuators in a pre-activation configuration.

FIG. 9A is an end view of the portion of the delivery device of FIG. 8, taken in the direction of arrow A in FIG. 8.

FIG. 9B is an end view of the portion of the delivery device of FIG. 8, taken in the direction of arrow B in FIG. 8.

FIG. 10A is the end view of the portion of the delivery device of FIG. 9A shown in an actuated configuration.

FIG. 10B is the end view of the portion of the delivery device of FIG. 9B shown in an actuated configuration.

FIG. 11 is a perspective view of the adaptor plate of FIG. 8.

FIG. 12 is a top view of the adaptor plate of FIG. 11.

FIG. 13 is a bottom view of the adaptor plate of FIG. 11.

FIG. 14 is a cross-sectional view of the adaptor plate of FIG. 11 taken along the line 14-14 in FIG. 12.

FIG. 15 is a perspective view of an electrochemical actuator of the delivery device of FIG. 5.

FIG. 16 is a side view of the electrochemical actuator of FIG. 15.

FIG. 17A is a schematic illustration of a top view of an adaptor plate according to an embodiment, and FIG. 17B is a schematic illustration of a bottom view of the adaptor plate of FIG. 17A.

FIG. 18A is a schematic illustration of a top view of an adaptor plate according to an embodiment; and FIG. 18B is a schematic illustration of a bottom view of the adaptor plate of FIG. 18A.

FIG. 19 is a perspective view of an adaptor plate according to an embodiment.

FIG. 20 is a perspective view of a portion of a delivery device according to an embodiment, including the adaptor plate of FIG. 19 and electrochemical actuators in an activated configuration.

FIG. 21 is a perspective view of a portion of a delivery device according to an embodiment, including adaptor clips and electrochemical actuators in a pre-activated configuration.

FIGS. 22A and 22B are side cross-sectional views of a portion of a delivery device according to an embodiment, including a cover, an adaptor, and electrochemical actuators in a pre-activated configuration and an activated configuration, respectively.

FIG. 23 is a perspective view of a portion of a delivery device according to an embodiment, including adaptor clips and electrochemical actuators in a pre-activated configuration.

FIG. 24 is a bottom perspective view of an adaptor clip of FIG. 23.

FIG. 25 is a perspective view of an adaptor clip according to an embodiment.

FIG. 26 is a perspective view of an adaptor clip according to an embodiment.

### DETAILED DESCRIPTION

Devices, systems and methods are described herein that are configured for use in the delivery of therapeutic agents to a patient's body. Such therapeutic agents can be, for example, one or more drugs and can be in fluid form of various viscosities. In some embodiments, the devices and methods can include a pump device that includes an actuator, such as, for example, an electrochemical actuator, which can have characteristics of both a battery and a pump. Specifically, an electrochemical actuator can include an electrochemical cell that produces a pumping force as the cell discharges. Thus, the pump device can have relatively fewer parts than a conventional drug pump, such that the pump device is relatively more compact, disposable, and reliable than conventional drug pumps. Such drug delivery devices are desirable, for example, for use in delivery devices that are designed to be attached to a patient's body (e.g., wearable device). These attributes of the pump device may reduce the cost and the discomfort associated with infusion drug therapy.

In some embodiments, such a pump device an be operated with, for example, a controller and/or other circuitry, operative to regulate drug or fluid flow from the pump device. Such a controller may permit implementing one or more release profiles using the pump device, including release profiles that require uniform flow, non-uniform flow, continuous flow, discontinuous flow, programmed flow, scheduled flow, user-initiated flow, or feedback responsive flow, among others. Thus, the pump device may effectively deliver a wider variety of drug therapies than other pump devices.

In some embodiments, a drug delivery system can include one or more actuators. For example the delivery system can include one or more electrochemical actuators. In some embodiments, one or more electrochemical actuators can be used in sequence or simultaneously. For example, in some embodiments, a first actuator can be actuated to provide a first phase of pumping at a first rate, and then a second actuator can be actuated to provide a second phase of pumping at a second rate, which may be the same as, or different than, the first rate. Thus, a combination of fast and slow delivery rates can be achieved. In some embodiments, a first electrochemical actuator may provide a faster rate of delivery than a second electrochemical actuator.

In some embodiments of a drug delivery system, a first electrochemical actuator and a second electrochemical actuator are used together to enhance the pumping force and/or duration and/or displacement capability of the delivery device. The use of multiple electrochemical actuators together, as described herein, can provide a mechanical advantage to the system operation. An adaptor, such as an adaptor plate or clips, can be used to allow for nesting or stacking of the electrochemical actuators. The use of multiple stacked actuators can increase the displacement capability of the delivery device when activated, thus allowing for an increased displacement rate, while maintaining the same force capabilities. For example, in some embodiments, the displacement can be doubled and the corresponding displacement rate can be doubled. Such enhanced pumping features can further increase the variety of different types of drug therapies that can be delivered using a wearable drug delivery system.

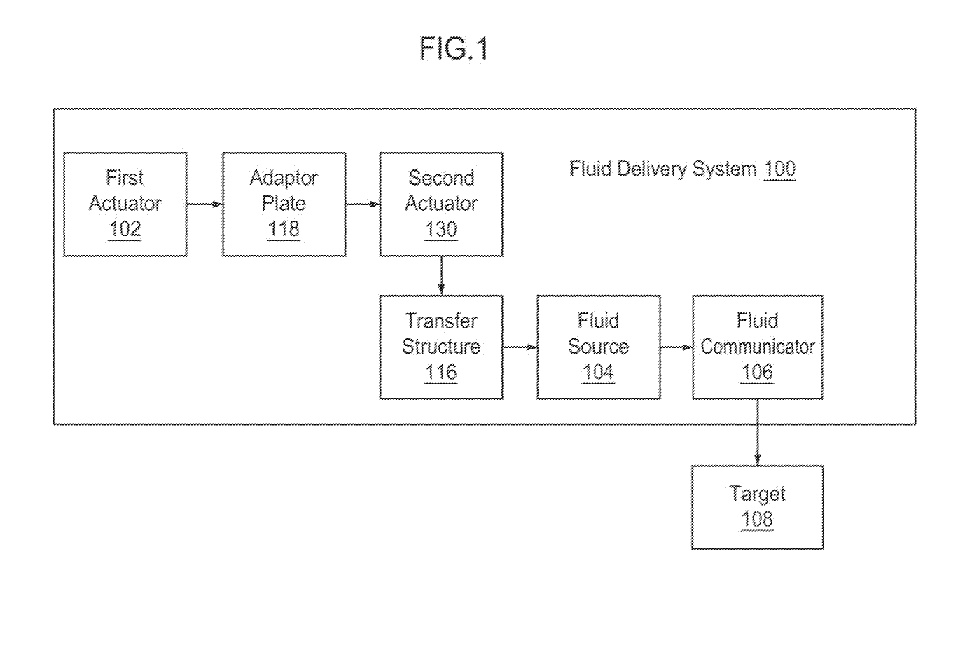

FIG. 1 is a schematic block diagram illustrating an embodiment of a fluid delivery system 100 (also referred to herein as “delivery device” or “drug delivery device”). The fluid delivery system 100 includes a first actuator 102, a second actuator 130, and adaptor 118, a transfer structure 116, a fluid source 104 and a fluid communicator 106. The fluid source 104 can contain a volume of fluid (e.g., a therapeutic agent) to be delivered into a target 108 via the fluid communicator 106. The target 108 can be, for example, a human or other mammalian body in need of a drug therapy or prophylaxis.

The first actuator 102 and the second actuator 130 can each be, for example, an electrochemical actuator that can actuate or otherwise create a pumping force to deliver the fluid from the fluid source 104 into the fluid communicator 106 as described in more detail below. In some embodiments, the first actuator 102 and the second actuator 130 can each be a device that experiences a change in volume or position in response to an electrochemical reaction that occurs therein. For example, the first actuator 102 and second actuator 130 can each be an electrochemical actuator that includes a charged electrochemical cell, and at least a portion of the electrochemical cell can actuate as the electrochemical cell discharges. Thus, the first actuator 102 and the second actuator 130 can each be considered a self-powered actuator or a combination battery and actuator.

As mentioned above, the use of multiple actuators can increase the displacement and/or force of the delivery system 100 to deliver a fluid volume that otherwise may not be possible without the use of multiple actuators. For example, with multiple actuators, a drug delivery device can, in some embodiments, achieve a longer stroke than with only a single actuator. A longer stroke can be leveraged to deliver larger drug doses, thus enabling new therapies previously not possible with known wearable drug delivery devices.

The adaptor 118 allows for nesting or stacking of the first actuator 102 (also referred to below as electrochemical actuator 102) and the second actuator 130 (also referred to below as electrochemical actuator 130). As mentioned above, the use of multiple stacked actuators can increase the displacement of the actuators when activated, thus allowing for an increased displacement rate of the delivery device 100, while maintaining the same force capabilities of the delivery device 100. The adaptor 118 can be an adaptor plate that can include a first recess defined in a first surface configured to receive a portion of the first actuator 102 and a second recess defined in a second surface opposite the first surface (e.g., on an opposite side of the adaptor plate 118) configured to receive a portion of the second actuator 130. In some embodiments, the first recess has a longitudinal axis that is orthogonal to a longitudinal axis of the second recess. Thus, when the first actuator 102 and the second actuator 130 are nested within their respective recesses, the first actuator 102 and the second actuator 130 are positioned orthogonal to each other. Such positioning of the actuators 102, 130 can provide vertical motion stability to the delivery device 100 during actuation, thereby eliminating the need to constrain the motion or otherwise provide for stability with other means. Such an embodiment is described in more detail below. In some embodiments, the first recess has a longitudinal axis that is parallel to a longitudinal axis of the second recess.

The first recess and the second recess can have a variety of different shapes and sizes configured to receive a portion of a corresponding actuator. In some embodiments, the first recess is substantially square shaped and the second recess is substantially rectangular shaped or vice versa. In some embodiments, both the first recess and the second recess are substantially square. In some embodiments, both the first recess and the second recess are substantially rectangular. In some embodiments, one or both of the first recess and second recess are circular, elliptical, oval, etc.

The fluid source 104 can be a reservoir, pouch, chamber, barrel, bladder, or other known device that can contain a drug in fluid form therein. The fluid communicator 106 can be in, or can be moved into, fluid communication with the fluid source 104. The fluid communicator 106 can be, for example, a needle, catheter, cannula, infusion set, or other known drug delivery conduit that can be inserted into or otherwise associated with the target body for drug delivery.

In some embodiments, the fluid source 104 can be any component capable of retaining a fluid or drug in fluid form. In some embodiments, the fluid source 104 may be disposable (e.g., not intended to be refillable or reusable). In other embodiments, the fluid source 104 can be refilled, which may permit reusing at least a portion of the device and/or varying the drug or fluid delivered by the device. In some embodiments, the fluid source 104 can be sized to correlate with the electrochemical potential of the electrochemical actuator 102 and/or the electrochemical actuator 130. For example, the size and/or volume of the fluid source 104 can be selected so that the fluid source 104 becomes about substantially empty at about the same time that the electrochemical actuator 102 and/or the electrochemical actuator 130 becomes about substantially discharged. By optimizing the size of the fluid source 104 and the amount of drug contained therein to correspond to the driving potential of the electrochemical actuators 102 and 130, the size and/or cost of the device may be reduced. In other embodiments, the electrochemical actuator 102 and/or the electrochemical actuator 130 can be oversized with reference to the fluid source 104. In some embodiments, the delivery system 100 can include more than one fluid source 104. Such a configuration may permit using a single device to deliver two or more drugs or fluids. The two or more drugs or fluids can be delivered discretely, simultaneously, alternating, according to a program or schedule, or in any other suitable manner. In such embodiments, the fluid sources 104 may be associated with the same or different electrochemical actuators 102 and 130, the same or different fluid communicators 106, the same or different operational electronics, or the same or different portions of other components of the delivery system. For example, the electrochemical actuator 102 can be configured to pump fluid out of a first fluid source and the electrochemical actuator 130 can be configured to pump fluid out of a second fluid source.

The transfer structure 116 can be disposed between the electrochemical actuator 102 and the fluid source 104 or between the electrochemical actuator 130 and the fluid source 104. The transfer structure 116 includes a surface configured to contact the fluid source 104 upon actuation of one or both of the actuators 102, 130 such that a force exerted by the electrochemical actuator 102 and/or electrochemical actuator 130 is transferred from the transfer structure 116 to the fluid source 104. The transfer structure 116 can include one or more components. For example, the transfer structure 116 can be a single component having a surface configured to contact the fluid source 104. In some embodiments, the transfer structure 116 can include one or more members having a surface configured to contact the fluid source 104 upon activation of the electrochemical actuator 102 and/or electrochemical actuator 130. In some embodiments, the transfer structure 116 is a substantially planar or flat plate. In some embodiments, a transfer structure 116 is not included. In such an embodiment, one or both of the electrochemical actuators 102, 130 can contact and exert a force directly upon the fluid source 104.

In some embodiments, the fluid delivery system 100 can be used to deliver a drug formulation which comprises a drug, including an active pharmaceutical ingredient. In other embodiments, the fluid delivery system 100 may deliver a fluid that does not contain a drug. For example, the fluid may be a saline solution or a diagnostic agent, such as a contrast agent. Drug delivery can be subcutaneous, intravenous, intraarterial, intramuscular, intracardiac, intraosseous, intradermal, intrathecal, intraperitoneal, intratumoral, intratympnic, intraaural, topical, epidural, and/or peri-neural depending on, for example, the location of the fluid communicator 106 and/or the entry location of the drug.

The drug (also referred to herein as “a therapeutic agent” or “a prophylactic agent”) can be in a pure form or formulated in a solution, a suspension, or an emulsion, among others, using one or more pharmaceutically acceptable excipients known in the art. For example, a pharmaceutically acceptable vehicle for the drug can be provided, which can be any aqueous or non-aqueous vehicle known in the art. Examples of aqueous vehicles include physiological saline solutions, solutions of sugars such as dextrose or mannitol, and pharmaceutically acceptable buffered solutions, and examples of non-aqueous vehicles include fixed vegetable oils, glycerin, polyethylene glycols, alcohols, and ethyl oleate. The vehicle may further include antibacterial preservatives, antioxidants, tonicity agents, buffers, stabilizers, or other components.

Although the fluid delivery system 100 and other systems and methods described herein are generally described as communicating drugs into a human body, such systems and methods may be employed to deliver any fluid of any suitable biocompatibility or viscosity into any object, living or inanimate. For example, the systems and methods may be employed to deliver other biocompatible fluids into living beings, including human beings and other animals. Further, the systems and methods may delivery drugs or other fluids into living organisms other than human beings, such as animals and plant life. Also, the systems and methods may deliver any fluids into any target, living or inanimate.

The systems and methods described herein are generally systems and methods of delivering fluids using a delivery device 100 that includes an electrochemical actuator 102, such as a self-powered actuator and/or combined battery and actuator. Example embodiments of such electrochemical actuators are generally described in U.S. Pat. No. 7,541,715, entitled “Electrochemical Methods, Devices, and Structures” by Chiang et al., U.S. Patent Pub. No. 2008/0257718, entitled “Electrochemical Actuator” by Chiang et al., and U.S. Patent Pub. No. 2009/0014320, entitled “Electrochemical Actuator” by Chiang et al., and U.S. Pat. No. 7,828,771, entitled “Systems and Methods for Delivering Drugs” by Chiang et al. (“the '771 Patent”), the disclosure of each of which is incorporated herein by reference. Such electrochemical actuators can include at least one component that responds to the application of a voltage or current by experiencing a change in volume or position. The change in volume or position can produce mechanical work that can then act on a fluid source (e.g., fluid source 104) or may be transferred to a fluid source, such that a fluid can be delivered out of the fluid source.

In some embodiments, the electrochemical actuator 102 and/or electrochemical actuator 130 can each include a positive electrode and a negative electrode, at least one of which is an actuating electrode. These and other components of the electrochemical actuator can form an electrochemical cell, which can in some embodiments initially be charged. For example, the electrochemical cell may begin discharging when a circuit between the electrodes is closed, causing the actuating electrode to actuate. The actuating electrode can thereby perform work upon another structure, such as the fluid source, or a transfer structure associated with the fluid source, as described in more detail below. The work can then cause fluid to be pumped or otherwise dispensed from the fluid source into the target 108.

More specifically, the actuating electrode of the electrochemical actuator 102 (and/or electrochemical actuator 130) can experience a change in volume or position when the closed circuit is formed, and this change in volume or position can perform work upon the fluid source or transferring structure. For example, the actuating electrode may expand, bend, buckle, fold, cup, elongate, contract, or otherwise experience a change in volume, size, shape, orientation, arrangement, or location, such that at least a portion of the actuating electrode experiences a change in volume or position. In some embodiments, the change in volume or position may be experienced by a portion of the actuating electrode, while the actuating electrode as a whole may experience a contrary change or no change whatsoever. It is noted that the delivery device 100 can include more than two electrochemical actuators. For example, in some embodiments, the delivery device 100 can include one or more electrochemical actuators 102 arranged in series, parallel, or some combination thereof.

The delivery system 100 can also include a housing (not shown in FIG. 1) that can be removably or releasably attached to the skin of the patient. The various components of the delivery system 100 can be fixedly or releasably coupled to the housing. To adhere the delivery device 100 to the skin of a patient, a releasable adhesive can at least partially coat an underside of the housing. The adhesive can be non-toxic, biocompatible, and releasable from human skin. To protect the adhesive until the device is ready for use, a removable protective covering can cover the adhesive, in which case the covering can be removed before the device is applied to the skin. Alternatively, the adhesive can be heat or pressure sensitive, in which case the adhesive can be activated once the device is applied to the skin. Example adhesives include, but are not limited to, acrylate based medical adhesives of the type commonly used to affix medical devices such as bandages to skin. However, the adhesive is not necessary, and may be omitted, in which case the housing can be associated with the skin, or generally with the body, in any other manner. For example, a strap or band can be used.

The housing can be formed from a material that is relatively lightweight and flexible, yet sturdy. The housing also can be formed from a combination of materials such as to provide specific portions that are rigid and specific portions that are flexible. Example materials include plastic and rubber materials, such as polystyrene, polybutene, carbonate, urethane rubbers, butene rubbers, silicone, and other comparable materials and mixtures thereof, or a combination of these materials or any other suitable material can be used.

In some embodiments, the housing can include a single component or multiple components. In some embodiments, the housing can include two portions: a base portion and a movable portion. The base portion can be suited for attaching to the skin. For example, the base portion can be relatively flexible. An adhesive can be deposited on an underside of the base portion, which can be relatively flat or shaped to conform to the shape of a particular body area. The movable portion can be sized and shaped for association with the base portion. In some embodiments, the two portions can be designed to lock together, such as via a locking mechanism. In some cases, the two portions can releasably lock together, such as via a releasable locking mechanism, so that the movable portion can be removably associated with the base portion. To assemble such a housing, the movable portion can be movable with reference to the base portion between an unassembled position and an assembled position. In the assembled position, the two portions can form a device having an outer shape suited for concealing the device under clothing. Various example embodiments of a housing are described in the '771 Patent.

The size, shape, and weight of the delivery device 100 can be selected so that the delivery device 100 can be comfortably worn on the skin after the device is applied via the adhesive. For example, the delivery device 100 can have a size, for example, in the range of about 1.0″×1.0″×0.1″ to about 5.0″×5.0″×1.0″, and in some embodiments in a range of about 2.0″×2.0″×0.25″ to about 4.0″×4.0″×0.67″. The weight of the delivery device 100 can be, for example, in the range of about 5 g to about 200 g, and in some embodiments in a range of about 15 g to about 100 g. The delivery device 100 can be configured to dispense a volume in the range of about 0.1 ml to about 1,000 ml, and in some cases in the range of about 0.3 ml to about 100 ml, such as between about 0.5 ml and about 5 ml. The shape of the delivery device 100 can be selected so that the delivery device 100 can be relatively imperceptible under clothing. For example, the housing can be relatively smooth and free from sharp edges. However, other sizes, shapes, and/or weights are possible.

As mentioned above, the electrochemical actuator 102 and the electrochemical actuator 130 can be used to cause the fluid delivery device 100 to delivery a drug-containing or non-drug containing fluid into a human patient or other target 108. Such a fluid delivery system 100 can be embodied in a relatively small, self-contained, and disposable device, such as a patch device that can be removably attached to the skin of a patient as described above. The delivery device 100 can be relatively small and self-contained, in part because, the electrochemical actuators 102 and 130 server as both the battery and a pump. The small and self-contained nature of the delivery device 100 advantageously may permit concealing the device beneath clothing and may allow the patient to continue normal activity as the drug is delivered. Unlike conventional drug pumps, external tubing to communicate fluid from the fluid reservoir into the body can be eliminated. Such tubing can instead be contained within the delivery device, and a needle or other fluid communicator can extend from the device into the body. The electrochemical actuator 102 and electrochemical actuator 130 can initially be charged, and can begin discharging once the delivery device 100 is activated to pump or otherwise deliver the drug or other fluid into the target 108. Once the electrochemical actuator 102 and the electrochemical actuator 130 have completely discharged or the fluid source 104 (e.g. reservoir) is empty, the delivery device 100 can be removed. The small and inexpensive nature of the electrochemical actuators 102, 130 and other components of the device may, in some embodiments, permit disposing of the entire device after a single use. The delivery device 100 can permit drug delivery, such as subcutaneous or intravenous drug delivery, over a time period that can vary from several minutes to several days. Subsequently, the delivery device 100 can be removed from the body and discarded.

In use, the delivery device 100 can be placed in contact with the target 108 (e.g. placed on the surface of a patient's body), such that the fluid communicator 106 (e.g., a needle, cannula, etc.) is disposed adjacent to a desired injection site. The fluid communicator 106 can be actuated with the actuation of the electrochemical actuator 102 or separately as described in more detail below. For example, the delivery device 100 can include a separate mechanism to actuate the fluid communicator 106. Activation of the fluid communicator 106 can include, for example, insertion of the fluid communicator 106 into the patient's body. Example embodiments illustrating various configurations for actuation of the fluid communicator 106 are described in the '771 Patent incorporated by reference above. The electrochemical actuator 102 and the electrochemical actuator 130 can then be actuated, simultaneously or sequentially, to apply a force or forces on the fluid source 104, causing the fluid to be delivered through the fluid communicator 106 and into the target 108. For example, as the electrochemical actuator 102 and electrochemical actuator 130 are actuated, the actuator 102 and the actuator 130 will each be displaced and one or both of the actuators 102, 130 (depending on the particular configuration) will contact and apply a force to the transfer structure 116. That force will in turn be transferred from the transfer structure 116 to the fluid source 104 to pump the fluid out of the fluid source 104, through the fluid communicator 106, and into the target 108.

Having described above various general principles, several exemplary embodiments of these concepts are now described. These embodiments are only examples, and many other configurations of a delivery system and/or the various components of a delivery system, are contemplated.

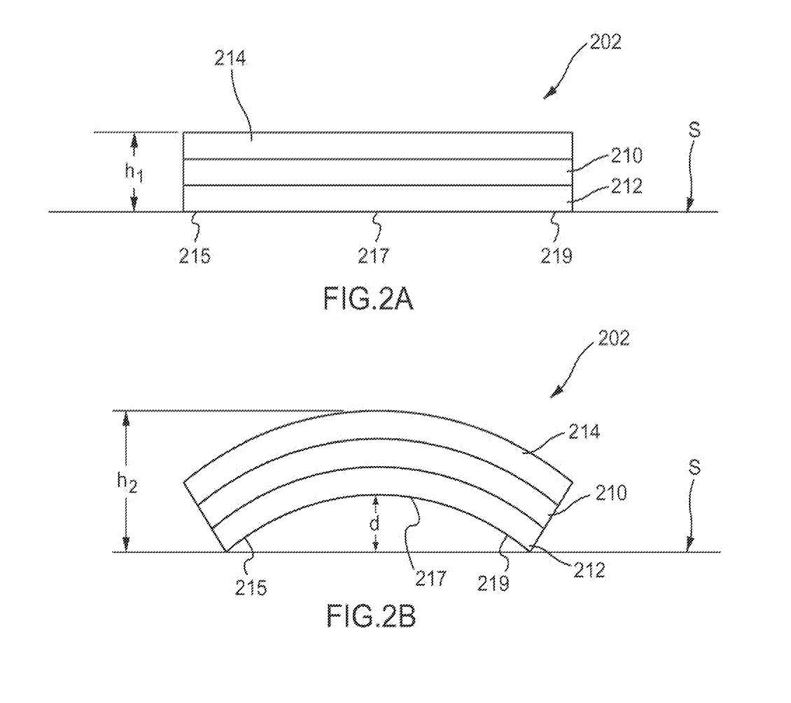

FIGS. 2A and 2B are schematic illustrations of an embodiment of an electrochemical actuator 202 that can be used in a delivery device as described herein. As shown, in this embodiment, the electrochemical actuator 202 can include a positive electrode 210, a negative electrode 212, and an electrolyte 214. These components can form an electrochemical cell that can initially be discharged and then charged before use, or can be initially charged, as shown in FIG. 2A. The positive electrode 210 can be configured to expand or displace in the presence of the electrolyte 214. When a circuit between the electrodes 210, 212 is closed, current can travel from the positive electrode 210 to the negative electrode 212. The positive electrode 210 can then experience a change in volume or shape, resulting in longitudinal displacement of at least a portion of the positive electrode 210, as shown in FIG. 2B. For example, the actuator 202 can have an overall height h₁ when it is charged (prior to actuation), as shown in FIG. 2A, and an overall height of h₂ when it is discharged or actuated, such that the actuator 202 has a displacement or stroke that is equal to h₂−h₁. Said another way, the actuator 202 can have a first end portion 215, a second end portion 219 and a medial portion 217 disposed between the first end portion 215 and the second end portion 219. The actuator prior to actuation (prior to discharge) can be supported on a surface S of the delivery device in which the actuator 202 is disposed, and when the actuator 202 is discharged at least the medial portion 217 can displace (e.g., bend or flex) a non-zero distance from the surface S. The stroke of the actuator 202 can be substantially equal to that non-zero distance. As the actuator 202 is displaced, the actuator 202 can exert a pumping force or pressure on a fluid reservoir (not shown) and/or an associated transfer structure (not shown) coupled thereto. The pumping force or pressure exerted by the actuator 202 can cause a volume of fluid (e.g., a therapeutic agent) to be pumped out of the fluid reservoir. Thus, the electrochemical actuator 202 can be considered a self-powered electrochemical pump.

In this embodiment, the electrochemical actuator 202 has a positive electrode 210 selected to have a lower chemical potential for the working ion when the electrochemical actuator 202 is charged, and is thereby able to spontaneously accept working ions from the negative electrode 212 as the actuator is discharged. In some embodiments, the working ion can include, but is not limited to, the proton or lithium ion. When the working ion is lithium, the positive electrode 210 can include one or more lithium metal oxides including, for example, LiCoO₂, LiFePO₄, LiNiO₂, LiMn₂O₄, LiMnO₂, LiMnPO₄, Li₄Ti₅O₁₂, and their modified compositions and solid solutions; oxide compound comprising one or more of titanium oxide, manganese oxide, vanadium oxide, tin oxide, antimony oxide, cobalt oxide, nickel oxide or iron oxide; metal sulfides comprising one or more of TiSi₂, MoSi₂, WSi₂, and their modified compositions and solid solutions; a metal, metal alloy, or intermetallic compound comprising one or more of aluminum, silver, gold, boron, bismuth, gallium, germanium, indium, lead, antimony, silicon, tin, or zinc; a lithium-metal alloy; or carbon comprising one or more of graphite, a carbon fiber structure, a glassy carbon structure, a highly oriented pyrolytic graphite, or a disordered carbon structure. The negative electrode 212 can include, for example, lithium metal, a lithium metal alloy, or any of the preceding compounds listed as positive electrode compounds, provided that such compounds when used as a negative electrode are paired with a positive electrode that is able to spontaneously accept lithium from the negative electrode when the actuator is charged. These are just some examples, as other configurations re also possible.

In some embodiments, the electrochemical actuator can include an anode, a cathode, and a species, such as a lithium ion. In some embodiments, a source of lithium ion is the electrolyte which is made up an organic solvent such as PC, propylene carbonate, GBL, gamma butyl lactone, dioxylane, and others, and an added electrolyte. Some example electrolytes include LiPF₆, LiBr, LiBF₄. At least one of the electrodes can be an actuating electrode that includes a first portion and a second portion. The portions can have at least one differing characteristic, such that in the presence of a voltage or current, the first portion responds to the species in a different manner than the second portion. For example, the portions can be formed from different materials, or the portions can differ in thickness, dimension, porosity, density, or surface structure, among others. The electrode can be charged, and when the circuit is closed, current can travel. The species can, intercalate, de-intercalate, alloy with, oxide, reduce, or plate with the first portion to a different extent than the second portion. Due to the first portion responding differently to the species than the second portion, the actuating electrode can experience a change in one or more dimensions, volume, shape, orientation, or position.

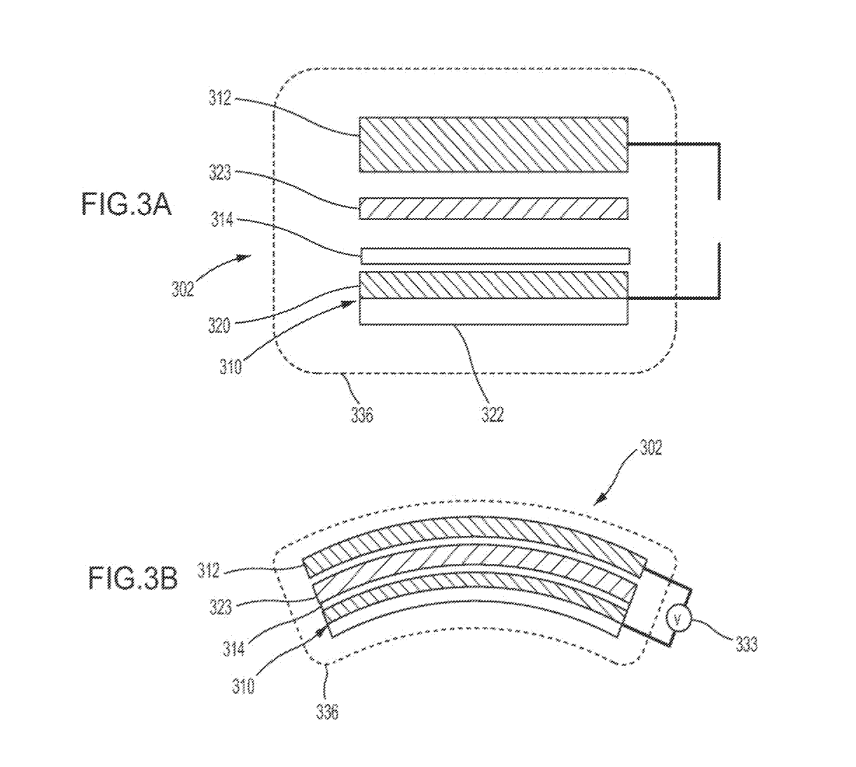

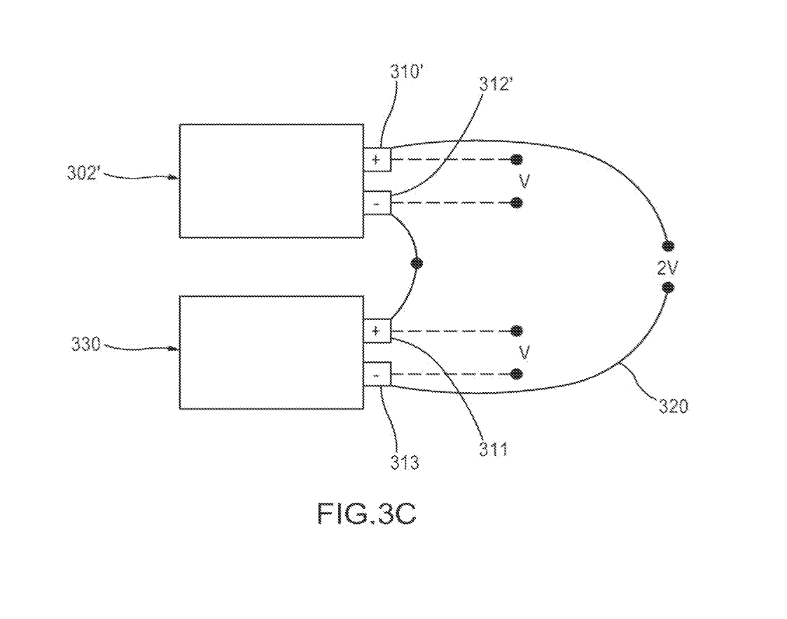

Another example of an electrochemical actuator is shown in the embodiment illustrated in FIGS. 3A and 3B. As shown in FIG. 3A, an electrochemical actuator 302 can include a negative electrode 312 in electrical communication with a positive electrode 310 collectively forming an electrochemical cell. Positive electrode 31 may include a first portion 320 and a second portion 322. In some embodiments, first portion 320 and second portion 322 are formed of different materials. Portions 320 and 322 may also have different electrical potentials. For example, first portion 320 may include a material that can intercalate, de-intercalate, alloy with, oxidize, reduce, or plate a species to a different extent than second portion 322. Second portion 322 may be formed of a material that does not substantially intercalate, de-intercalate, or alloy with, oxidize, reduce, or plate the species. In some embodiments, first portion 320 may be formed of a material including one or more of aluminum, antimony, bismuth, carbon, gallium, silicon, silver, tin, zinc, or other materials which can expand upon intercalation or alloying or compound formation with lithium. In one embodiment, first portion 320 is formed with aluminum, which can expand upon intercalation with lithium. Second portion 322 may be formed of copper, since copper does not substantially intercalate or alloy with lithium. In some instances, second portion 322 may act as a positive electrode current collector, and may extend outside the electrochemical cell, e.g., to form a tab or current lead. In other embodiments, second portion 322 may be joined to a tab or current lead that extends outside the cell. Negative electrode 312 may also include a current collector. Electrochemical actuator 302 may include a separator 323. The separator 323 may be, for example, a porous separator film, such as a glass fiber cloth, or a porous polymer separator. Other types of separators, such as those used in the construction of lithium ion batteries, may also be used. The electrochemical actuator 302 may also include an electrolyte 314, which may be in the form of a liquid, solid, or a gel. The electrolyte may contain an electrochemically active species, such as that used to form the negative electrode. Electrochemical actuator 302 may also include an enclosure 336, such as a polymer packaging, in which negative electrode 312, positive electrode 310 and separator 323 can be disposed.

As illustrated in FIG. 3B, the electrochemical cell may have a voltage 333, such that, when a closed circuit is formed between the negative electrode 312 and the positive electrode 310, an electric current may flow between the negative electrode 312 and the positive electrode 310 through the external circuit. If negative electrode 312 is a lithium metal electrode and the electrolyte contains lithium ions, lithium ion current can flow internally from the negative electrode 312 to the positive electrode 310. The intercalation of first portion 320 with lithium can result in a dimensional change, such as a volume expansion. In some instances, this volume expansion may reach at least 25%, at least 50%, at least 75%, at least 100%, at least 150%, at least 200%, at least 250%, or at least 300% compared to the initial volume. High volume expansion may occur, for example, when first portion 320 is saturated with lithium. As first portion 320 increases in volume due to intercalation of lithium, second portion 322 to which first portion 320 may be bonded, may not substantially expand due to minimal or no intercalation of lithium. First portion 320 thus provides a mechanical constraint. This differential strain between the two portions causes positive electrode 310 to undergo bending or flexure. As a result of the dimensional change and displacement of the positive electrode 310, electrochemical actuator 302 can be displaced from a first orientation to a second orientation. this displacement can occur whether the volumetric or dimensional change (e.g., net volume change) of the electrochemical cell, due to the loss of lithium metal from the negative electrode 312 and formation of lithium intercalated compound or lithium alloy at the positive electrode 310, is positive, zero, or negative. In some cases, the actuator displacement may occur with a volumetric or dimensional change (e.g., net volume change) of the electrochemical actuator 302, or portion thereof that is positive. In some cases, the actuator displacement may occur with a volumetric or dimensional change (e.g., net volume change) of the electrochemical actuator 302, or portion thereof that is zero. In some cases, the actuator displacement may occur with a volumetric or dimensional change (e.g., net volume change) of the electrochemical actuator 302, or portion thereof that is negative.

As used herein, “differential strain” between two portions can refer to the difference in response (e.g., actuation) of each individual portion upon application of a voltage or current to the two portions. That is, a system as described herein may include a component including a first portion and a second portion associated with (e.g., may contact, may be integrally connected to) the first portion, wherein, under essentially identical conditions, the first portion may undergo a volumetric or dimensional change and the second portion does not undergo a volumetric or dimensional change, producing strain between the first and second portions. The differential strain may cause the component, or a portion thereof, to be displaced from a first orientation to a second orientation. In some embodiments, the differential strain may be produced by differential intercalation, de-intercalation, alloying, oxidation, reduction, or plating of a species with one or more portions of the actuator system.

For example, the differential intercalation, de-intercalation, alloying, oxidation, reduction, or plating of first portion 320 relative to second portion 322 can be accomplished through several means. In one embodiment, first portion 320 may be formed of a different material than second portion 322, wherein one of the materials substantially intercalates, de-intercalates, alloys with, oxidizes, reduces, or plates a species, while the second portion interacts with the species to a lesser extent. In another embodiment, first portion 320 and second portion 322 may be formed of the same material. For example, first portion 320 and second portion 322 may be formed of the same material and may be substantially dense, or porous, such as a pressed or sintered powder or foam structure. In some cases, to produce a differential strain upon operation of the electrochemical cell, first portion 320 or second portion 322 may have sufficient thickness such that, during operation of the electrochemical cell, a gradient in composition may arise due to limited ion transport, producing a differential strain. In some embodiments, one portion or an area of one portion may be preferentially exposed to the species relative to the second portion or area of the second portion. In other instances, shielding or masking of one portion relative to the other portion can result in lesser or greater intercalation, de-intercalation, or alloying with the masked or shielded portion compared to the non-masked or shielded portion. This may be accomplished, for example, by a surface treatment or a deposited barrier layer, lamination with a barrier layer material, or chemically or thermally treating the surface of the portion to be masked/shielded to either facilitate or inhibit intercalation, de-intercalation, alloying, oxidation, reduction, or plating with the portion. Barrier layers can be formed of any suitable material, which may include polymers, metals, or ceramics. In some cases, the barrier layer can also serve another function in the electrochemical cell, such as being a current collector. The barrier layer may be uniformly deposited onto the surface in some embodiments. In other cases, the barrier layer may form a gradient in composition and/or dimension such that only certain portions of the surface preferentially facilitate or inhibit intercalation, de-intercalation, alloying, oxidation, reduction, or plating of the surface. Linear, step, exponential, and other gradients are possible. In some embodiments a variation in the porosity across first portion 320 or second portion 322, including the preparation of a dense surface layer, may be used to assist in the creation of an ion concentration gradient and differential strain. Other methods of interaction of a species with a first portion to a different extent so as to induce a differential strain between the first and second portions can also be used. In some embodiments, the flexure or bending of an electrode is used to exert a force or to carry out a displacement that accomplishes useful function.

In some embodiments, the electrical circuit can include electrical contacts (not shown) that can open or close the electrical circuit. For example, when the electrical contacts are in communication with each other, the electrical circuit will be closed (as shown in FIG. 3B) and when they are not in contact with each other, the electrical circuit can be opened or broken, as shown in FIG. 3A.

The discharge of the electrochemical actuator can be relatively proportional to the current traveling through the electrical circuit (i.e., the electrical resistance of the resistor). Because the electrical resistance of the resistor can be relatively constant, the electrochemical actuator can discharge at a relatively constant rate. Thus, the discharge of the electrochemical actuator, and thus the displacement of the electrochemical actuator can be relatively linear with the passage of time.

In some embodiments, an electrical circuit can be used that includes a variable resistor. By varying the resistance, the discharge rate of the electrochemical actuator and the corresponding displacement of the electrochemical actuator can be varied, which in turn can vary the fluid flow rate from the fluid source. An example of such an embodiment is described in the '771 Patent. In some embodiments, an electrical circuit can be used that uses a switch to open or close the electrical circuit. When the switch is closed, the electrochemical actuator can discharge and when the switch is opened, the electrochemical actuator can be prevented from discharging. An example of such an embodiment is described in the '771 Patent incorporated by reference above.

Although the foregoing discussion describes an electrical circuit formed between electrodes (e.g., 310, 312) of a single electrochemical actuator 302, in some embodiments, an electrical circuit can be formed between electrodes of multiple electrochemical actuators. For example, as schematically illustrated in FIG. 3C, an electrical circuit 320 can be used that includes a first electrochemical actuator 302′ and a second electrochemical actuator 330. Each of the electrochemical actuators 302′, 330 can be similar in many respects to electrochemical actuator 302 described above, except as noted herein.

Specifically, a positive electrode 310′ of the first actuator 302′ is in electrical communication with a negative electrode 313 of the second actuator 330, and a negative electrode 312′ of the first actuator 302′ is in electrical communication with a positive electrode 311 of the second actuator 330. As such, whereas the electrochemical cell described above with reference to FIGS. 3A and 3B has a voltage 333 when a closed circuit is formed between its negative electrode 312 and its positive electrode 310, when a closed circuit is formed between the negative electrode 312′ of the first electrochemical actuator 302 and the positive electrode 311 of the second electrochemical actuator 330 and between the negative electrode 313 of the second electrochemical actuator 330 and the positive electrode 310′ of the first electrochemical actuator 302, as in the embodiment of FIG. 3C, a combined voltage 2V substantially equal to at least the sum of the voltage potential V of each electrochemical actuator 302′, 330 is produced.

For example, if each electrochemical actuator 302′, 330 has a voltage potential V substantially equal to the voltage 333 of the electrochemical cell described above, when the electric circuit 320 is closed between the electrodes of the electrochemical actuators 302′, 330, the electrical circuit has a voltage of about two times voltage 333. In another example, the first electrochemical actuator 302′ can have a voltage V of about 0.3 and the second electrochemical actuator 330 can have a voltage V of about 0.3. Because the first and second electrochemical actuators 302′, 330 are included in the single (or same) electrical circuit 320, the effective or total voltage 2V of the circuit is about 0.6. In this manner, the displacement of each of the first and second electrochemical actuators 302′, 330 can be greater in the presence of the total voltage 2V of the electrical circuit 320, for example, than would otherwise occur in the presence of the voltage V (e.g., an electrical circuit with a single actuator). Additionally, the electrochemical actuators 302′, 330 can collectively produce sufficient power to drive electronic components of a delivery system which a single electrochemical actuator may have insufficient power to drive.

Although the electrochemical actuators 302′, 330 are described as being about 0.3 volts individually, and 0.6 volts collectively, in other embodiments, each electrochemical actuators 302′, 330 can have any suitable voltage. Furthermore, the electrochemical actuators 302′, 330 can have the same voltage, or different voltages. Although the circuit 320 has been illustrated and described as including two electrochemical actuators 302′, 330, in other embodiments, an electrical circuit can include three or more electrochemical actuators. Additionally, the electrochemical actuators 302′, 330 can be connected in parallel, effectively doubling the capacity (amp hours) of the electrochemical actuators 302′, 330 while maintaining the voltage of the electrical circuit at that of a single electrochemical actuator.

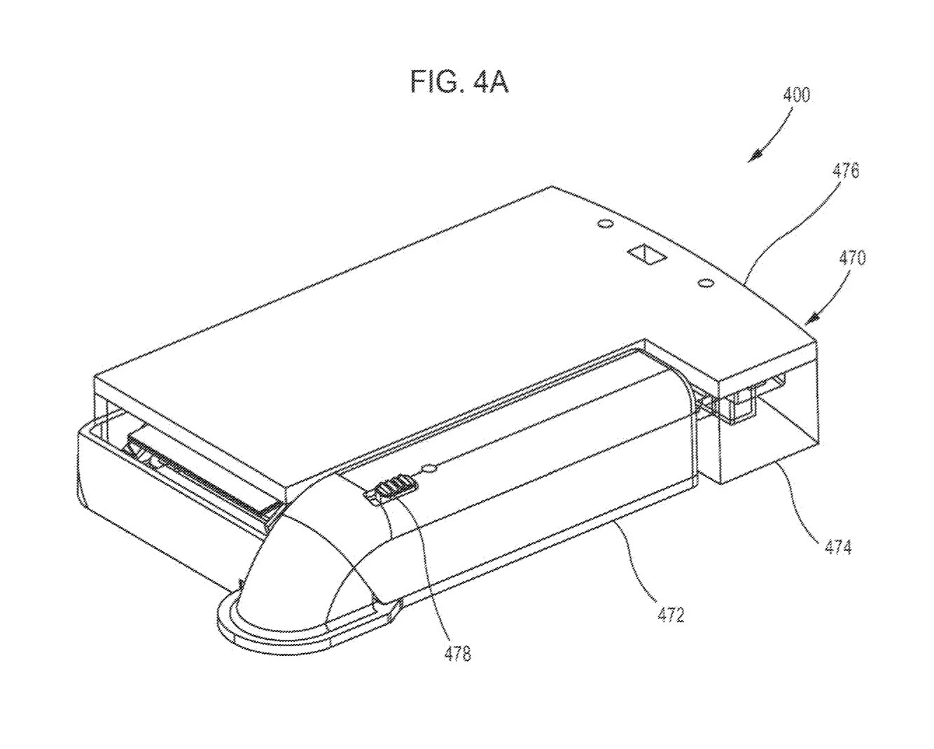

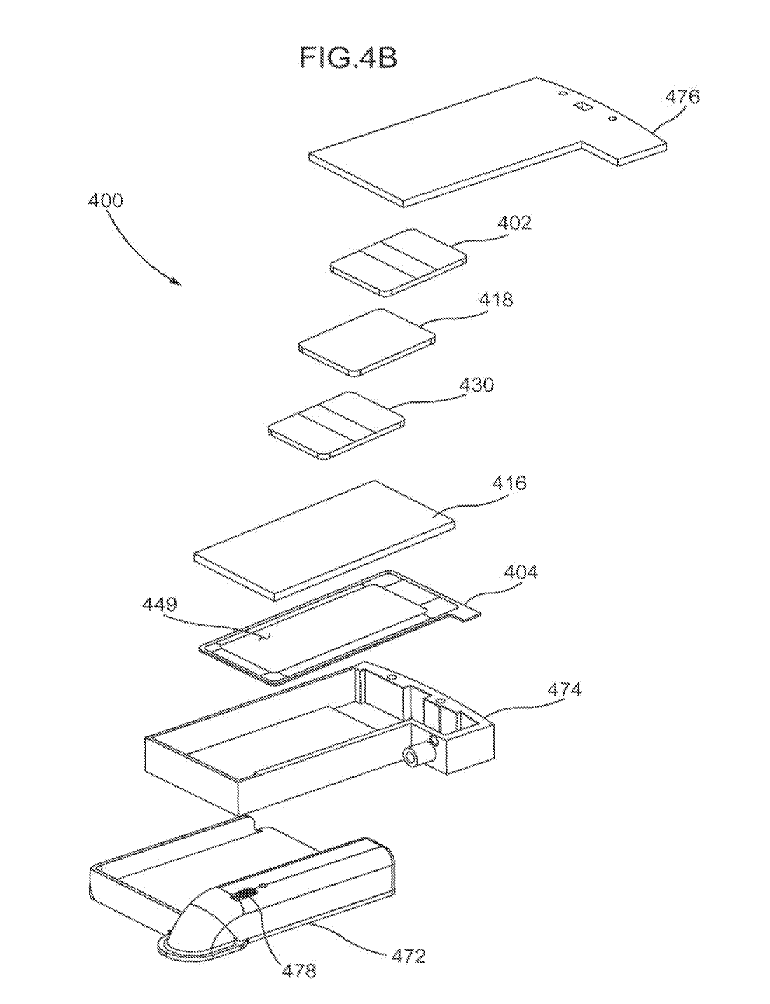

FIGS. 4A and 4B illustrate an embodiment of a delivery device that can include two electrochemical actuators as described herein. A delivery device 400 includes a housing 470, a fluid source 404, electrochemical actuators 402, 430, an adaptor 418 (shown schematically in FIG. 4B), optionally, a transfer structure 416 can be disposed between the fluid source 404 and the actuators 402, 430, and associated electronics (not shown) that can be coupled to the electrochemical actuators 402, 430. In this embodiment, the housing 470 includes a first portion 472, a second portion 474, and a top portion 476 that can be coupled together to form an interior region within the housing 470. The fluid source 404, the electrochemical actuators 402, 430, the adaptor 418 and the transfer structure 416 can each be disposed within the interior region defined by the housing 470.

The fluid source 404 can be provided to a user predisposed within the interior region of the housing 470 or can be provided as a separate component that the user can insert into the housing 470. For example, the fluid source 404 can be inserted through an opening (not shown) in the housing 470. The fluid source 404 can be, for example, a fluid reservoir, bag or container, etc. that defines an interior volume that can contain a fluid to be injected into a patient. The fluid source 404 (also referred to herein as “fluid reservoir”) can include a web portion (not shown) configured to be punctured by an insertion mechanism (not shown) to create a fluid channel between the fluid source 404 and a fluid communicator (not shown) configured to penetrate the patient's skin. In some embodiments, the fluid reservoir 404 can be sized for example, with a length L of about 2 cm, a width W of about 2 cm, and a height H of about 0.25 cm, to contain, for example, a total volume of 1 ml of fluid.

The delivery device 400 also includes an activation mechanism 478 in the form of button that can be used to activate the insertion mechanism and/or one or more of the actuators 402, 430. The first portion 472, the second portion 474 and the top portion 476 of the housing 470 can be coupled together in a similar manner as with various embodiments of a delivery system described in the '771 Patent incorporated by reference above. The first portion 472, the second portion 474 and the top portion 476 can be coupled, for example, with an adhesive, a snap fit coupling or other known coupling method. The first portion 472 can be adhered to a patient's body with an adhesive layer disposed on a bottom surface of the first portion 472.

To use the delivery device 400, the delivery device 400 is placed at a desired injection site on a patient's body and adhesively attached thereto. When the fluid source 404 is disposed within the housing 470 (e.g., inserted into the housing by the patient or predisposed), the activation mechanism 478 (e.g., button, switch, lever, pull-tab, etc.) can be moved from an off position to an on position, which will cause the fluid communicator to penetrate the patient's skin at the treatment site. Alternatively, in some embodiments, the insertion mechanism (not shown) can be activated by the fluid source 404 being inserted into the housing.

The electrochemical actuators 402, 430 can be activated after the insertion mechanism has been activated and the fluid communicator is inserted into the patient's body. Alternatively, in some embodiments, the electrochemical actuators 402, 430 can be activated simultaneously with activation of the insertion mechanism. For example, when the insertion mechanism is activated it can be configured to activate a trigger mechanism (not shown) that communicates with at least one of the electrochemical actuators 402, 430. For example, such a trigger mechanism can complete the electric circuit (as described above) and cause at least one of the electrochemical actuators 402, 430 to start discharging. As the electrochemical actuator 402 discharges, the actuators 402, 430 and the adaptor 418 will displace and exert a force on the transfer structure 416, which in turn will exert a force on the top surface 449 of the fluid source 404, thereby compressing the fluid source 404 between the transfer structure 416 and the second portion 474 of the housing 470 and causing a volume of fluid within the fluid source 404 to be expelled into the patient. Similarly, as the electrochemical actuator 430 discharges, the actuator 430 will displace and exert a force on the transfer structure 416, which in turn will exert a force on the top surface 449 of the fluid source 404, thereby compressing the fluid source 404 between the transfer structure 416 and the second portion 474 of the housing 470 and causing a volume of fluid within the fluid source 404 to be expelled into the patient. In some embodiments, the electrochemical actuators 402, 430 can be discharged simultaneously (or at overlapping periods of time), and thus both actuators 402, 430 and the adaptor 418 will displace and exert a combined force on the transfer structure 416. The transfer structure 416, in turn, will exert the combined force on the top surface 449 of the fluid source 404, thereby compressing the fluid source 404 between the transfer structure 416 and the second portion 474 of the housing and causing a volume of fluid within the fluid source 404 to be expelled into the patient.

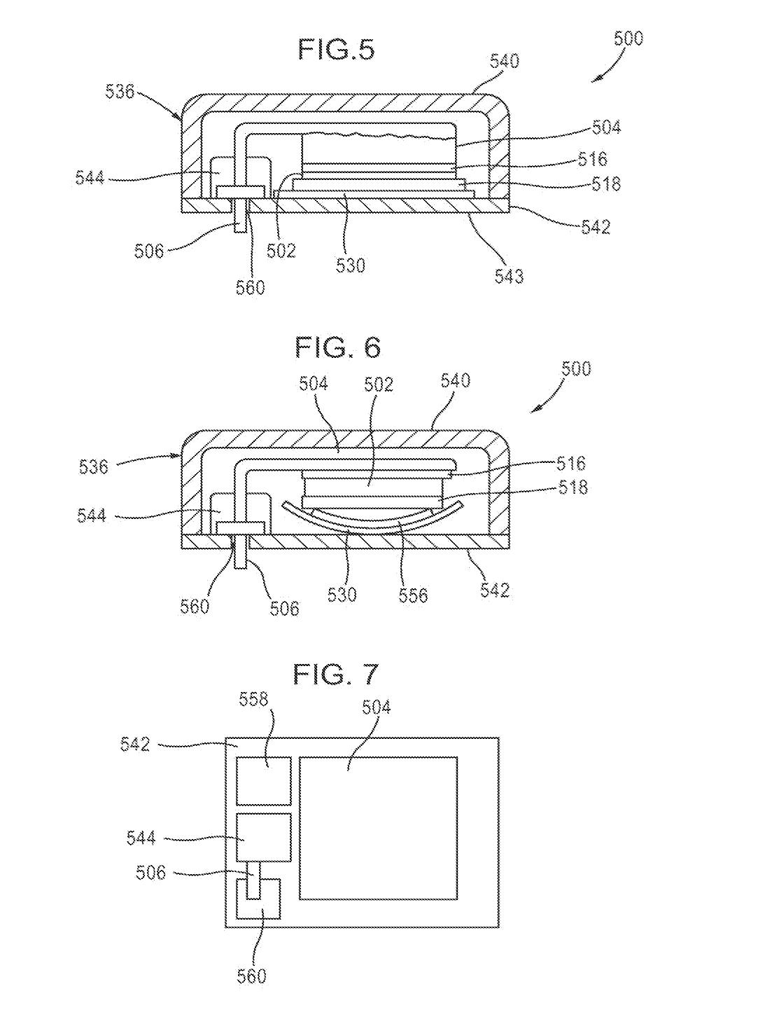

FIGS. 5-16 illustrate an embodiment of a drug delivery device that includes two electrochemical actuators and an adaptor plate. A delivery device 500 includes a housing 536, a fluid source 504, a fluid communicator 506, a first electrochemical actuator 502, a second electrochemical actuator 530, an adaptor plate 518, a transfer structure 516, a fluid communicator insertion mechanism 544, and associated electronics 558 (see, e.g., FIG. 7).

The housing 536 includes an upper wall portion 540 and a lower wall portion 542 that can be coupled together in a similar manner as with various embodiments of a delivery system described in the '771 Patent. For example, the upper wall portion 540 can be snapped or locked onto the bottom wall portion 542. In some embodiments, the upper wall portion 540 and the bottom wall portion 542 can be adhesively coupled together. The upper wall portion 540 and the bottom wall portion 542 collectively define an interior region of the housing 536 in which various components of the delivery device 500 are disposed. The lower wall portion 542 can be adhered to a patient's body with an adhesive layer disposed on a bottom surface 543 of the bottom wall portion 542.

In this embodiment, the fluid communicator 506 is in the form of a cannula that can be inserted into a patient's body using the insertion mechanism 544. For example, the insertion mechanism 544 can be configured to insert the fluid communicator 506 through an opening 560 defined in the lower wall portion 542 of the housing 536. The insertion mechanism 544 can be configured in a similar manner as with various embodiments described in the '771 Patent. In alternative embodiments, a separate insertion mechanism can be used. The fluid communicator 506 can be placed in fluid communication with the fluid reservoir 506 such that it can communicate the fluid within the fluid reservoir 504 to the patient. For example, the insertion mechanism 544 can be configured to puncture the fluid reservoir 504 upon activation to create a fluid path between the fluid reservoir 504 and the fluid communicator 506.

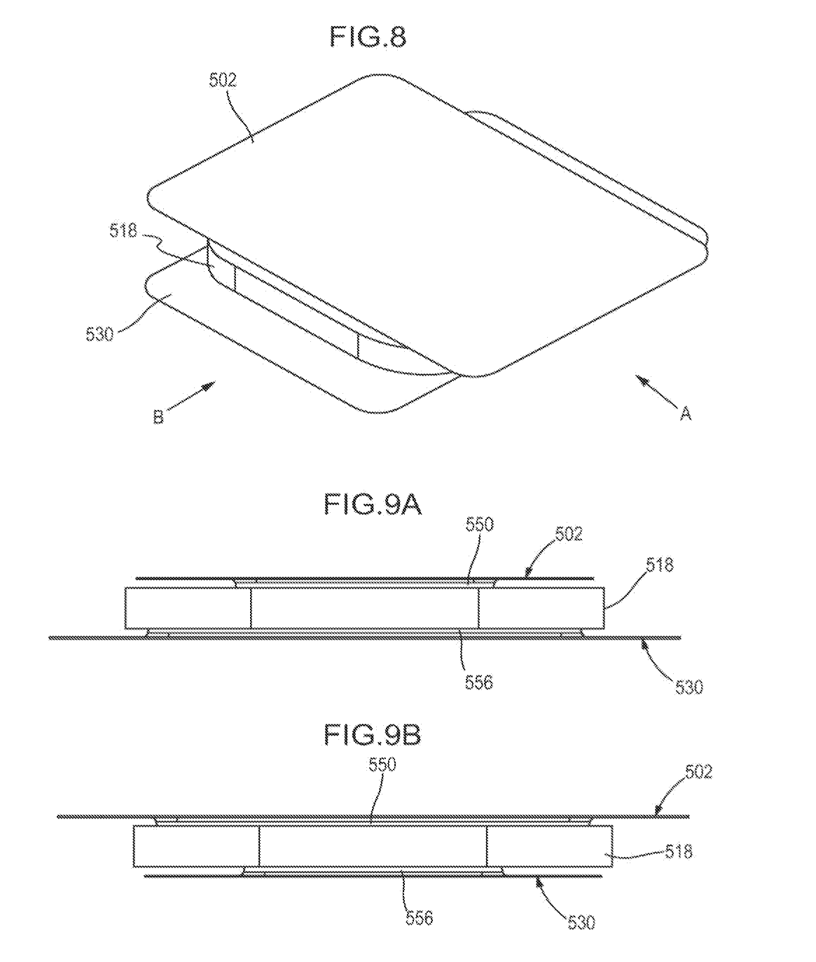

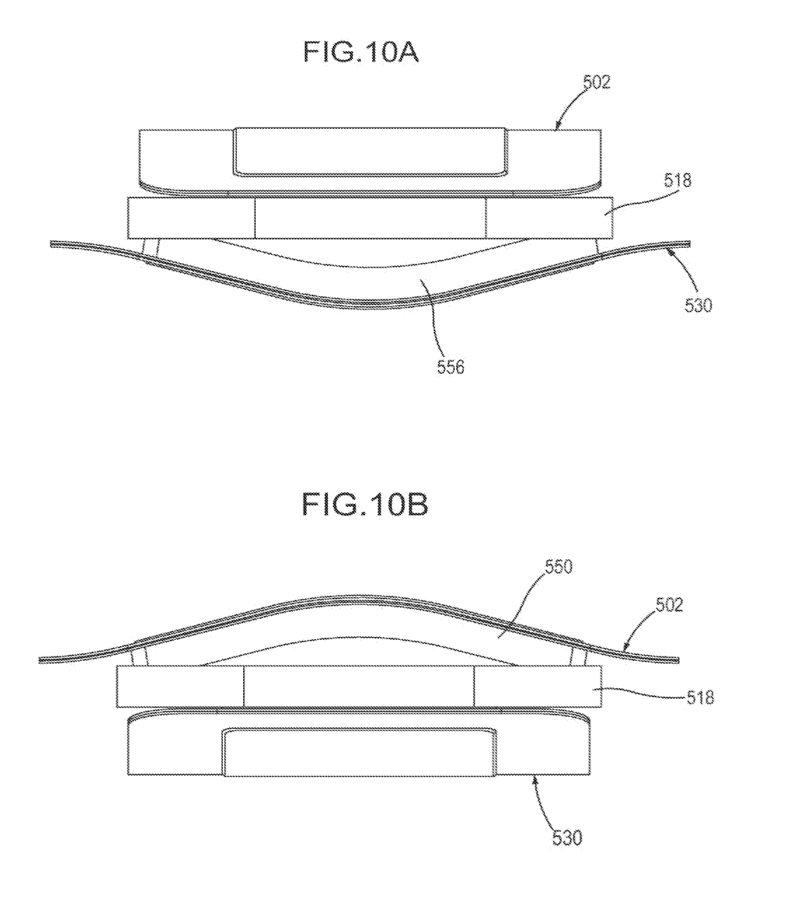

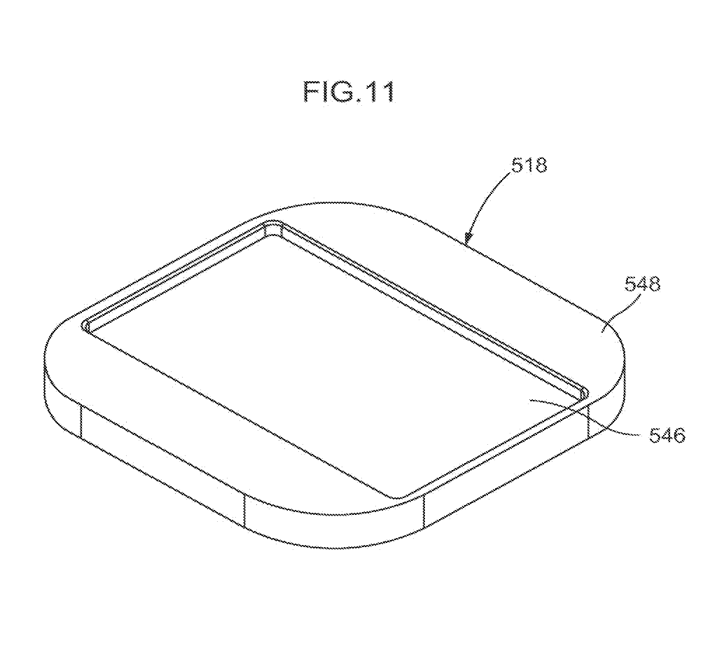

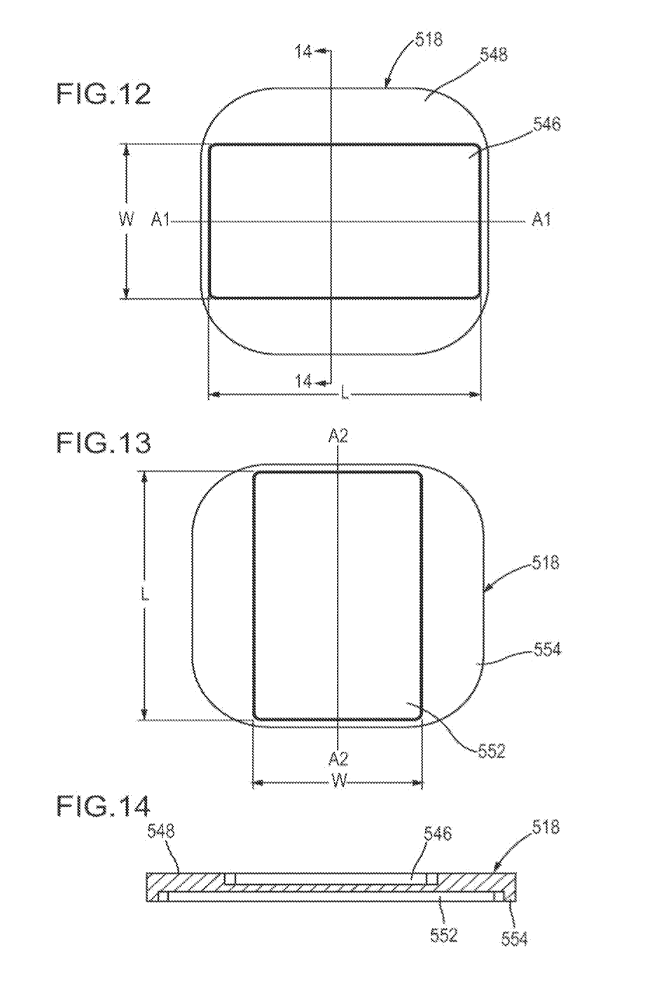

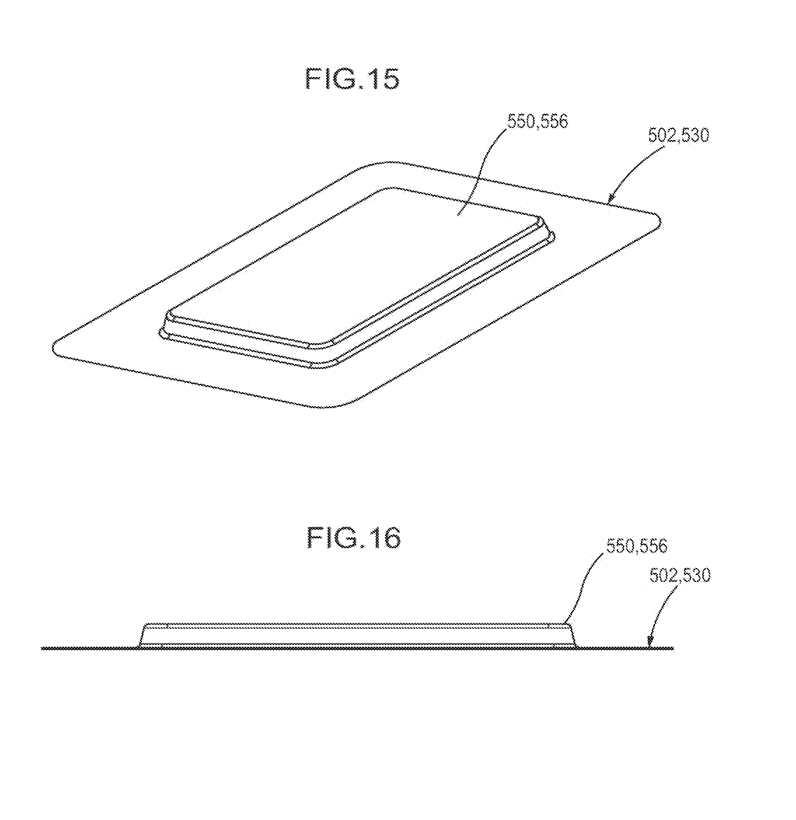

FIGS. 8-11 illustrate the coupling of the first actuator 502 and the second actuator 530 to the adaptor plate 518. As shown in FIG. 8, in this embodiment, the first actuator 502 is coupled to the adaptor plate 518 orthogonally to the second actuator 530. This relationship can be further viewed in FIGS. 9A through 10B. FIGS. 9A and 9B illustrate the actuators 502 and 530 in a pre-activated or charged configuration and FIGS. 10A and 10A illustrate the actuators 502 and 530 in an activated or expanded configuration. FIGS. 9A and 10B are each an end view in the direction of Arrow A in FIG. 8, and FIGS. 9B and 10B are each an end view in the direction of Arrow B in FIG. 8. As shown in FIGS. 11 and 12, the adaptor plate 518 defines a first recess or pocket 546 in a top surface 548 that is configured to receive a raised portion 550 (see e.g., FIGS. 15 and 16) of the first actuator 502. The adaptor 518 also defines a second recess or pocket 552 defined in a bottom surface 554 as shown in FIG. 13 (see also the cross-sectional view of FIG. 14). The first recess 546 defines a first longitudinal axis A1 and the second recess 552 defines a second axis A2. In this embodiment, the first axis A1 is transverse to axis A2. In some embodiments, the longitudinal axis A1 of the first recess 546 can be orthogonal to the longitudinal axis A2 of the second recess 552. The first recess 546 and the second recess 552 each have a length L and a width W, as shown in FIGS. 12 and 13.

Adaptor plate 518 can be various shapes and or sizes and the first recess 546 and the second recess 552 can have a variety of different shapes and sizes. In some embodiments, the first recess 546 has the same shape and/or size as the second recess 552 and in some embodiments, the first recess 546 and the second recess 552 can have different shapes and/or sizes. For example, for the adaptor plate 518, the length L and width W is the same for the first recess 546 and the second recess 552. In one embodiment, the length L can be, for example, about 41.25 mm, and the width W can be, for example, 25.15 mm. In another example, the length L can be about 29.55 mm and the width W can be about 25.55 mm.

In this embodiment, the first actuator 520 and the second actuator 530 are constructed the same and are illustrated in FIGS. 15 and 16. As shown, the first actuator 502 and the second actuator 530 each include a raised portion 550 and 556, respectively, configured to be received within the first recess 546 and the second recess 552, respectively. Thus, when nested within the adaptor plate 518, the first actuator 502 and the second actuator 530 are oriented transverse to relative to each other as shown, for example, in FIG. 8. Such positioning of the actuators 502, 530 provides vertical motion stability to the delivery device 500 during actuation, thereby eliminating the need to constrain the motion or otherwise provide for stability with other means. In some embodiments, the first actuator 502 and the second actuator 530 are oriented orthogonally relative to each other. In some embodiments, however, the recesses 546, 552 and the actuators 502, 530 can be positioned in alignment and other accommodations can be provided to adjust for possible tilting of the actuators during activation of the delivery device. Thus, as mentioned previously, in some embodiments, the longitudinal axis A1 of the first recess 546 can be parallel to and/or in alignment with, the longitudinal axis A2 of the second recess 552.

In use, the delivery device 500 can be attached to a patient's body, for example, by adhesively attaching the bottom surface 543 of the lower wall portion 542 of the housing 536 to the skin of the patient. The insertion mechanism 544 can be activated to insert the fluid communicator 506 into the patient. Activation of the insertion mechanism 544 can be achieved, for example, by actuating an activation mechanism (not shown) that can be a switch, button, pull-tab, etc. The insertion mechanism 544 can also be used to trigger activation of one or both of the electrochemical actuators 502 and 530 upon insertion of the fluid communicator 506. In some embodiments, a secondary activation mechanism (not shown) is provided to start activation of the actuators 502, 530.

Referring back to FIGS. 5 and 6, FIG. 8 illustrates the delivery device 500 when the electrochemical actuator 502 and the electrochemical actuator 530 are both in a charged state (pre-activation), and the fluid communicator 506 has been inserted into the patient's body. In this configuration, the delivery device 500 is in a ready mode. As described above, the electrochemical actuators 502, 530 can be triggered to begin discharging upon insertion of the fluid communicator 506 or with a secondary mechanism. As described previously, when the electrochemical actuator 502 and the electrochemical actuator 530 are activated (e.g. the electrochemical actuators 502 and 530 are discharging), the actuator 502 and the actuator 530 will each be displaced or bend as shown in FIGS. 6, 10A and 10B. Specifically, during activation, the raised portion 550 of the first actuator 502 and the raised portion 556 of the second actuator 530 each move or displace in a direction away from the adaptor plate 518.

As the actuator 530 displaces or bends in a direction away from the adaptor plate 518, the actuator 530 will contact the bottom wall portion 542 of the housing 536 and push upward on the adaptor plate 518. As the actuator 502 displaces or bends in a direction away from the adaptor plate 518, the actuator 502 contacts the transfer structure 516 and causes it to move upward. The combined force caused by the displacement of the actuator 530 and the displacement of the actuator 502 will in turn exert a force to the fluid reservoir 504, squeezing the fluid reservoir 504 between the transfer structure 516 and the upper wall portion 540 of the housing 536. The fluid in the fluid reservoir 504 will be pumped or expelled out of the fluid reservoir 504, through the fluid communicator 506 and into the patient's body.

As discussed previously, the first actuator 502 and the second actuator 530 can be configured to be activated sequentially or simultaneously. Thus, if activated sequentially, a first displacement of one of the actuators can result in a first force being applied to the fluid source 504 and then a second displacement of the other actuator can result in a second force to be applied to the fluid source 504. If activated simultaneously, a combined displacement of both the actuators will cause a force to be exerted on the fluid source 504 at an increased displacement rate.

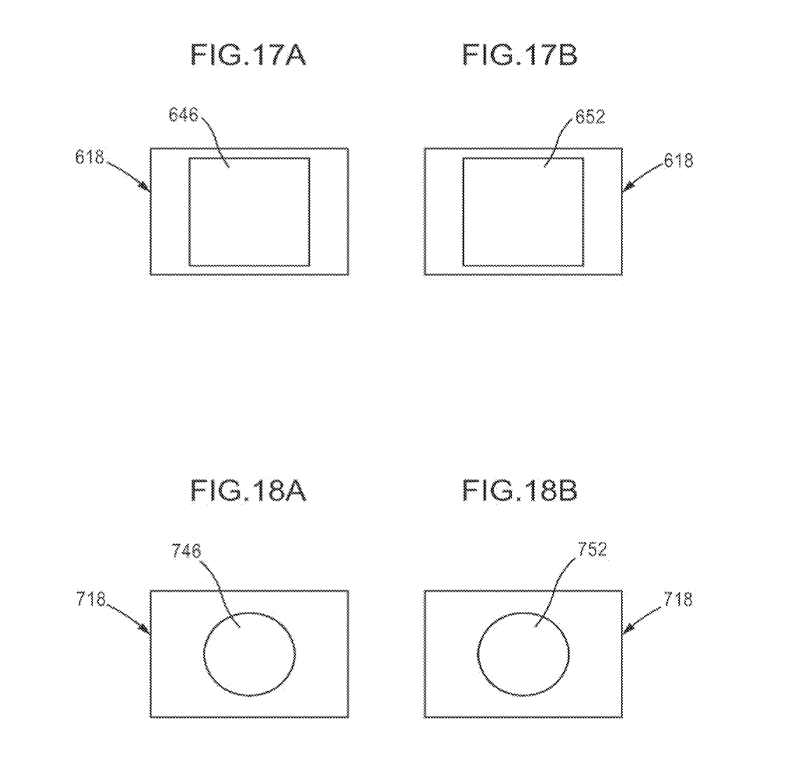

FIGS. 17A and 17B are schematic illustrations of another embodiment of an adaptor that can be used to nest multiple electrochemical actuators as described herein. FIG. 17A is a plan view of a top side of an adaptor plate 618, and FIG. 17B is a plan view of a bottom side of the adaptor plate 618. The adaptor plate 618 defines a first recess 646 and a second recess 652 that are each substantially square shaped and each the same or substantially the same size.

FIGS. 18A and 18B are schematic illustrations of another embodiment of an adaptor plate that can be used to nest multiple electrochemical actuators as described herein. FIG. 18A is a plan view of a top side of an adaptor plate 718, and FIG. 18B is a plan view of a bottom side of the adaptor plate 718. The adaptor 718 defines a first recess 746 and a second recess 752 that are circular shaped and each are the same or substantially the same size.

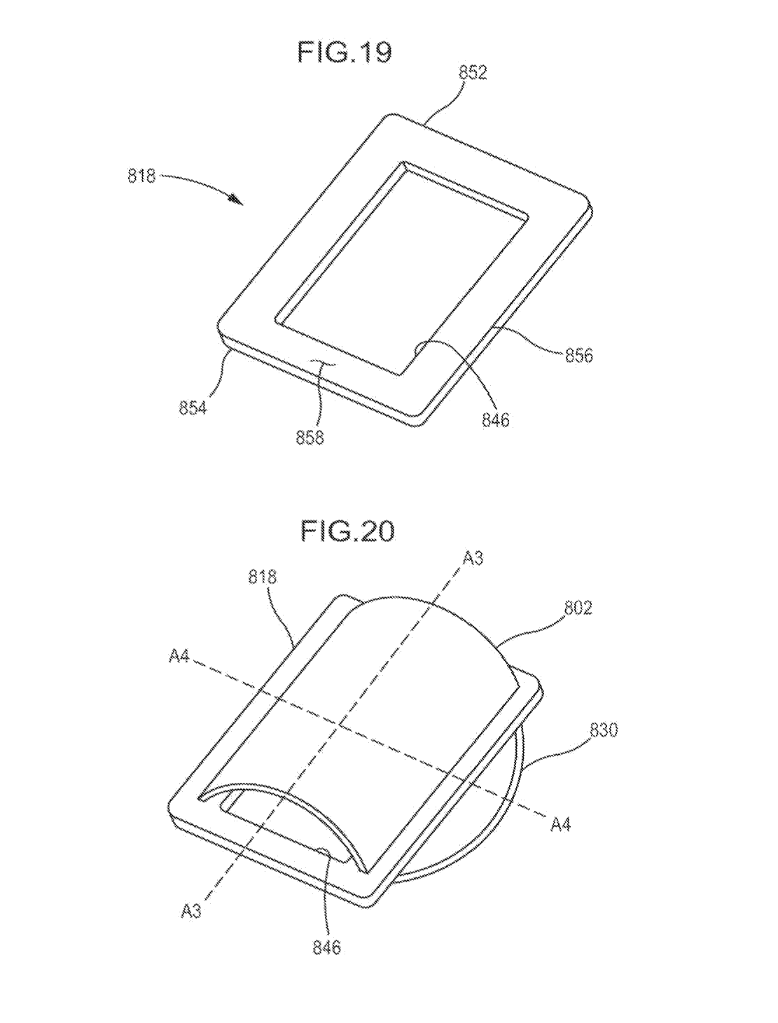

Although an adaptor plate (e.g., adaptor plate 518, 618, 718) has been illustrated and described herein as defining a first recess (e.g., first recess 546, 646, 746) and a second recess (e.g., second recess 552, 652, 752), in some embodiments, an adaptor plate can be configured differently. For example, as illustrated in FIG. 19, an adaptor plate 818 includes a first end portion 852, a second end portion 854, and a medial portion 856. an aperture 846 is defined by the medial portion 856 of the adaptor plate 818. The aperture 846 is extended between a first surface 858 and a second surface (not shown) of the adaptor plate 818. As such, the adaptor plate 818 can have a reduced weight compared to an adaptor plate with a solid medial portion. In some embodiments, the aperture 846 of the adaptor plate 818 can have similar length and/or width dimensions as described above with respect to recesses 546, 552 of adaptor plate 518.

A first actuator 802 is disposed adjacent the first surface of the adaptor plate 818. A second actuator 830 is disposed adjacent the second surface of the adaptor plate 818. The actuators 802, 830 can be disposed with respect to the adaptor plate 818 in any manner described herein, including with respect to adaptor plates 518, 618, 718. For example, as illustrated in FIG. 20, the first actuator 802 defines a longitudinal axis A3 and the second actuator 830 defines a longitudinal axis A4. In some embodiments, the longitudinal axis A3 of the first actuator 802 is transverse, or even orthogonal, to the longitudinal axis A4 of the second actuator 830. In other embodiments, the longitudinal axis A3 of the first actuator 802 and the longitudinal axis A4 of the second actuator 830 can be substantially parallel.

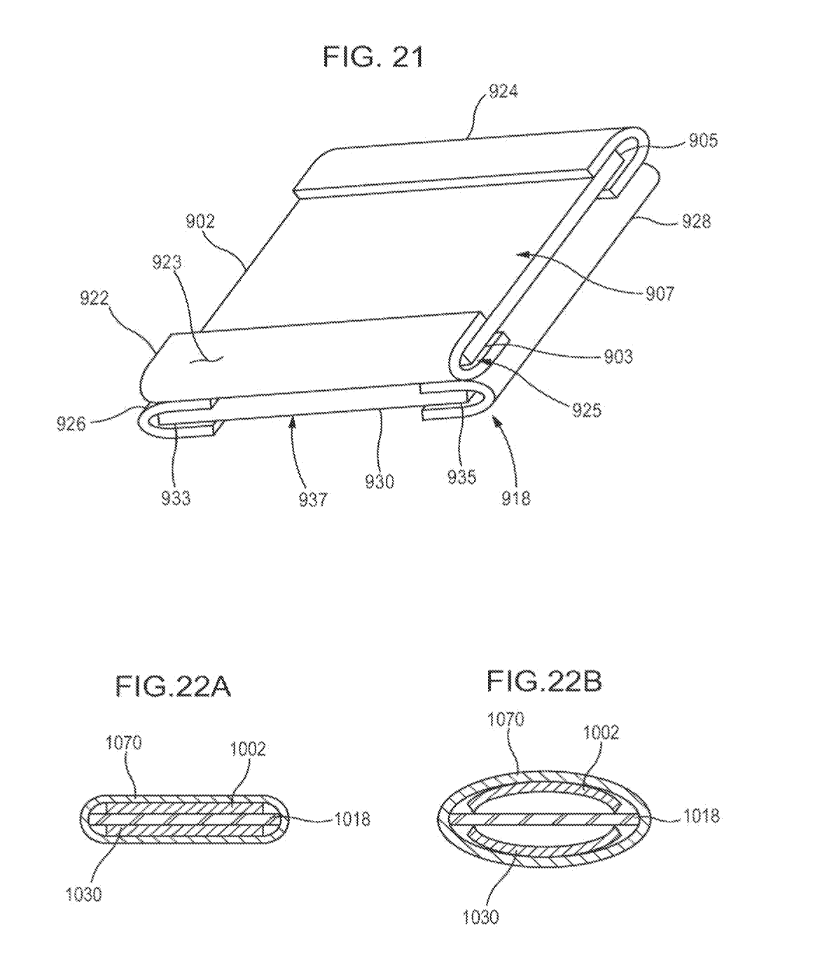

Although the adaptor (e.g., adaptor plate 518, 618, 718, 818) has been illustrated and described herein as being in the form of an adaptor plate, in other embodiments, an adaptor can include one or more clips. For example, referring to FIG. 21, a drug delivery device can include a first actuator 902, an adaptor 918, and a second actuator 930. The first actuator 902 can be similar in many respects to any actuator described herein (e.g., actuator 102, 202, 302, 402, 502, 602, 702, 802). The second actuator 930 can be similar in many respects to any actuator described herein (e.g., actuator 130, 230, 330, 430, 530, 630, 730, 830).

The adaptor 918 is configured to coupled the first actuator 902 and the second actuator 930. The adaptor 918 includes one or more clips 922, 924, 926, 928. In the embodiment illustrated in FIG. 21, each clip 922, 924, 926, 928 has an upper surface 923 and a lower surface (not shown) and defines a channel 925 therebetween. The first clip 922 is disposed about a first end portion 903 of the first actuator 902 such that the first end portion is received in the channel of the first clip. The second clip 924 is disposed about a second end portion 905 of the first actuator such that the second end portion is received in the channel of the second clip. Similarly, the third clip 926 is disposed about a first end portion 933 of the second actuator 930 such that the first end portion is received in the channel of the third clip and the fourth clip 928 is disposed about a second end portion 935 of the second actuator such that the second end portion is received in the channel of the fourth clip. The clips 922, 924, 926, 928 can be coupled to its respective actuator 902, 930 in any suitable manner, including, for example, by a friction fit, an adhesive, a mechanical fastener, or the like.

The first actuator 902 and its clips 922, 924 are disposed on the second actuator 930 and its clips 926, 928 (e.g., in a stacked configuration). Because the first actuator 902 is positioned orthogonal to the second actuator 930, the first and second clips 922, 924 are also positioned orthogonal to the third and fourth clips 926, 928. As such, as shown in FIG. 21, at least a portion of the first clip 922 is engaged with at least a portion of each of the third clip 926 and the fourth clip 928. Similarly, at least a portion of the second clip 924 is engaged with at least a portion of each of the third clip 926 and the fourth clip 928. In some embodiments, the adaptor 918 includes an interlocking mechanism (not shown) configured to interlock at least one of the first actuator 902 clips 922, 924 to at least one of the second actuator 930 clips 926, 928. The interlocking mechanism can be configured to maintain alignment of the first actuator 902 with respect to the second actuator 930, for example, such that the first actuator does not twist or become off-centered from its stacked configuration with respect to the second actuator. The interlocking mechanism can include any suitable mechanism to movably couple a first actuator 902 clip 922, 924 to a second actuator 930 clip 926, 928, including, for example, mating recesses, a locking pin and complementary slot, or the like.

In use, prior to actuation of the first actuator 902 (e.g., when the first actuator is in a first configuration such as its inactivated shape), the first actuator is substantially flat or planar. When the first actuator 902 is actuated, a portion of the first actuator is moved in a direction away from the second actuator 930 that is substantially perpendicular to its flat configuration (e.g., a second configuration of the first actuator). For example, when the first actuator 902 is in its second configuration, the medial portion 907 of the first actuator 902 can be offset from the first actuator's 902 inactivated plane. As the first actuator 902 is moved from its first configuration towards its second configuration, and the medial portion 907 of the first actuator is displaced, the first clip 922 and the second clip 924 are moved towards each other. In so moving, the clips 922, 924 each slide against (or are otherwise engaged with) the clips 926, 928 of the second actuator. In a similar manner, the second actuator 930 is movable from a first configuration in which the second actuator is substantially flat or planar to a second configuration in which a portion of the second actuator is deformed substantially perpendicular to its first (i.e., flat) configuration. For example, when the second actuator 930 is in its second configuration, its medial portion 937 can offset from the second actuator's inactivated plane. As the second actuator 930 is moved towards its second configuration, the medial portion 937 is moved in a direction away from the first actuator 902, and the clips 926, 928 of the second actuator are moved towards each other, each sliding against the clips 922, 924 of the first actuator 902.

The clips 922, 924, 926, 926 are each configured to substantially prevent secondary displacement, or bending, of the actuators 902, 930. For example, the clips 922, 924 disposed about the end portions 903, 905 of the first actuator 902 substantially prevent the first actuator from bending in a direction that would be complementary to (or a mirror-image of) the bending of the medial portion 937 of the second actuator 930. Similarly, in another example, the clips 926, 928 disposed about the end portions 933, 935 of the second actuator 930 substantially prevent the second actuator from bending in a direction that would be complementary to (or a mirror-image of) the bending of the medial portion 907 for the first actuator 902. In this manner, the clips 922, 924, 926, 928, are also configured to help redirect the displacement force during actuation of the respective actuators 902, 930 to the primary displacement (or bending) of the respective medial portion 907, 937, as described above. As such, the adaptor 918 can be characterized as being configured to permit displacement of an actuator (e.g., first actuator 902 or second actuator 930) in a first direction with respect to the adaptor, but substantially prevents displacement of the actuator in a second direction with respect to the adaptor.

Although the clips 922, 924, 926, 924 are each illustrated and described herein as having a length substantially equal to a length of its respective end portion 903, 905, 933, 935 of the actuators 902, 930, in other embodiments, one or more clips can have a length that is lesser than or greater than its respective actuator end portion.

In some embodiments, as illustrated in FIGS. 22A and 22B, a delivery device (not shown) can include a first actuator 1002, an adaptor 1018 (e.g., a plate and/or one or more clips), and a second actuator 1030 that are substantially enveloped by an enclosure or cover 1070. In this manner, the cover 1070 is configured to couple the first actuator 1002, the second actuator 1030, and the adaptor 1018. The cover 1070 is configured to retain the first actuator 1002 with respect to the adaptor 1018 and the second actuator 1030 when the first actuator is in a first configuration, as shown in FIG. 22A, and when the first actuator is in a second configuration during and/or after actuation, as shown in FIG. 22B. In other words, the cover 1070 can retain the first actuator 1002 with respect to the adaptor 1018 and the second actuator 1030 while permitting movement (e.g., deformation) of the first actuator during actuation. Similarly, the cover 1070 is configured to retain the second actuator with respect to the adaptor 1018 and the first actuator 1002 while permitting movement (e.g., deformation) of the second actuator during actuation. Specifically, the cover 1070 is configured to retain the second actuator 1030 with respect to the adaptor 1018 and the first actuator 1002 when the second actuator is in a first configuration, as shown in FIG. 22A, and when the second actuator is in a second configuration during and/or after actuation, as shown in FIG. 22B. The cover 1070 can be constructed of, for example, an elastomer or other suitable material.

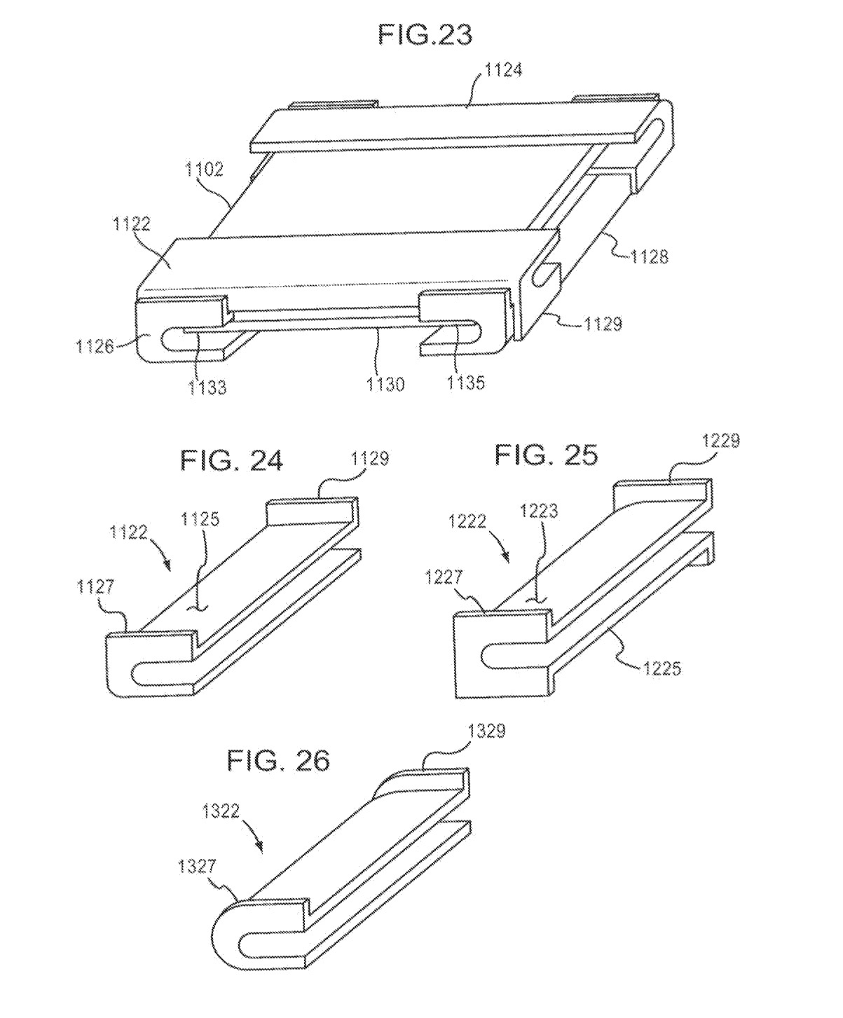

In some embodiments, a drug delivery device can include one or more adaptor clips that have an end portion configured to prevent unconstrained motion of a first actuator with respect to a second actuator. For example, referring to FIG. 23, a drug delivery device can include a first actuator 1102, a second actuator 1130, and clips 1122, 1124, 1126, 1128. The first actuator 1102 can be similar in many respects to any actuator described herein (e.g., actuator 102, 202, 302, 402, 502, 602, 702, 802, 902, 1002). The second actuator 1130 can be similar in many respects to any actuator described herein (e.g., actuator 130, 230, 330, 430, 530, 630, 730, 830, 930, 1030). The clips 1122, 1124, 1126, 1128 can be similar in many respects to the clips 922, 924, 926, 928 described above. For example, the clips 1122, 1124, 1126, 1128 are configured to couple the first actuator 1102 and the second actuator 1130, in a similar manner as described above with respect to clips 922, 924, 926, 928 and actuators 902, 930. The clips 1122, 1124 are disposed about opposing end portions (not shown in FIG. 23) of the first actuator 1102, and the clips 1126, 1128 are disposed about opposing end portions 1133, 1135 of the second actuator 1130.

Referring to FIGS. 23-24, the clip 1122 associated with the first actuator 1102 includes protrusions 1127, 1129 that extend from opposing end portions of the clip in a first direction away from or off of a lower surface 1125 of the clip (e.g., and not away from or off of an upper surface 1123 of the clip such that the protrusions can be characterized as being asymmetrical with respect to their respective end portions of the clip). In some embodiments, the protrusions 1127,1129 is orthogonal to a surface (e.g., lower surface 1125) of the clip 1122. The protrusions 1127, 1129 extend from the clip 1122 such that each protrusion is disposed adjacent one of the clips 1126, 1128 associated with the second actuator 1130. In some embodiments, at least one protrusion 1127, 1129, can engage the clip 1126, 1128, respectively, associated with the second actuator 1120. In this manner, the clips 1126, 1128 associated with the second actuator 1130 are substantially constrained between the protrusions 1127, 1129 of the clip 1122 associated with the first actuator 1102. Said another way, the protrusions 1127, 1129 of the clip 1122 are configured to limit movement of the clip 1128 in a direction away from the opposing end portion 1133 of the second actuator 1130 and/or clip 1126. As such, movement or displacement of the second actuator 1130 with respect to the first actuator 1102 is limited. Clips 1124, 1126, 1128 of the delivery device can also include similar protrusions, as illustrated in FIG. 23. As such, protrusions of clips 1126, 1128 associated with the second actuator 1130 are similarly configured to limit movement of the first actuator 1102 with respect to the second actuator.

Although the protrusions 1127, 1129 are illustrated and described herein as being asymmetrical with respect to their respective end portions of the clip 1122, in some embodiments, a clip can include a protrusion that is differently configured. For example, referring to FIG. 25, a clip 1222 can include protrusions 1227, 1229 that extend from opposing end portions of the clip 1222. Each protrusion 1227, 1229 extends away from a first surface 1223 and a second surface 1225 of the clip 1222. As such, the protrusions 1227, 1229 can each be characterized as being symmetrical with respect to its respective clip 1222 end portion.

Additionally, although clips (e.g., clip 1122, 1222) have been illustrated and described herein as including a protrusion (e.g., protrusion 1127, 1227) having a squared shape, in other embodiments, a clip can include a protrusion having any suitable shape. For example, in some embodiments, a clip 1322 can include protrusions 1327, 1329 that have a curved portion, as shown in FIG. 26. Embodiments are contemplated in which a clip can include various combinations of symmetrical and asymmetrical protrusions. It is also contemplated that a delivery device can include various combinations of clips with symmetrical and/or asymmetrical protrusions for limiting movement or displacement of the actuators with respect to each other.

A delivery device (e.g., 100, 500) as described herein may be used to deliver a variety of drugs according to one or more release profiles. For example, the drug may be delivered according to a relatively uniform flow rate, a varied flow rate, a preprogrammed flow rate, a modulated flow rate, in response to conditions sensed by the device, in response to a request or other input from a user or other external source, or combinations thereof. Thus, embodiments of the delivery device may be used to deliver drugs having a short half-life, drugs having a narrow therapeutic window, drugs delivered via on-demand dosing, normally-injected compounds for which other delivery modes such as continuous delivery are desired, drugs requiring titration and precise control, and drugs whose therapeutic effectiveness is improved through modulation delivery or delivery at a non-uniform flow rate. These drugs may already have appropriate existing injectable formulations.

For example, the delivery devices may be useful in a wide variety of therapies. Representative examples include, but are not limited to, opioid narcotics such as fentanyl, remifentanyl, sufentanil, morphine, hydromorphone, oxycodone and salts thereof or other opioids or non-opioids for post-operative pain or for chronic and breakthrough pain; NonSteroidal Antinflamatories (NSAIDs) such as diclofenac, naproxen, ibuprofin, and celecoxib; local anesthetics such as lidocaine, tetracaine, and bupivicaine; dopamine antagonists such as apormorphine, rotigotine, and ropinerole; drugs used for the treatment and/or prevention of allergies such as antihistamines, antileukotrienes, anticholinergics, and immunotherapeutic agents; antispastics such as tizanidine and baclofin; insulin delivery for Type 1 or Type 2 diabetes; leutenizing hormone releasing hormone (LHRH) or follicle stimulating hormone (FSH) for infertility; plasma-derived or recombinant immune globulin or its constituents for the treatment of immunodeficiency (including primary immunodeficiency), autoimmune disorders, neurological and neurodegenerative disorders (including Alzheimer's Disease), and inflammatory diseases; apomorphine or other dopamine agonists for Parkinson's disease; interferon A for chronic hepatitis B, chronic hepatitis C, solid or hematologic malignancies; antibodies for the treatment of cancer; octreotide for acromegaly; ketamine for pain, refractory depression, or neuropathic pain; heparin for post-surgical blood thinning; corticosteroid (e.g., prednisone, hydrocortisone, dexamethasone) for treatment of MS; vitamins such as niacin; Selegiline; and rasagiline. Essentially any peptide, protein, biologic, or oligonucleotide, among others, that is normally delivered by subcutaneous, intramuscular, or intravenous injection or other parenteral routes, may be delivered using embodiments of the devices described herein. In some embodiments, the delivery device can be used to administer a drug combination of two or more different drugs using a single or multiple delivery port and being able to deliver the agents at a fixed ratio or by means enabling the delivery of each agent to be independently modulated. For example, two or more drugs can be administered simultaneously or serially, or a combination (e.g. overlapping) thereof.

In some embodiments, the delivery device may be used to administer ketamine for the treatment of refractory depression or other mood disorders. In some embodiments, ketamine may include either the racemate, single enantiomer (R/S), or the metabolite (wherein S-norketamine may be active). In some embodiments, the delivery devices described herein may be used for administration of Interferon A for the treatment of hepatitis C. In one embodiment, a several hour infusion patch is worn during the day or overnight three times per week, or a continuous delivery system is worn 24 hours per day. such a delivery device may advantageously replace bolus injection with a slow infusion, reducing side effects and allowing the patient to tolerate higher doses. In other Interferon A therapies, the delivery device may also be used in the treatment of malignant melanoma, renal cell carcinoma, hairy cell leukemia, chronic hepatitis B, condylomata acuminata, follicular (non-Hodgkin's lymphoma, and AIDS-related Kaposi's sarcoma.

In some embodiments, a delivery device as described herein may be used for administration of apomorphine or other dopamine agonists in the treatment of Parkinson's Disease (“PD”). Currently, a bolus subcutaneous injection of apomorphine may be used to quickly jolt a PD patient out of an “off” state. However, apomorphine has a relatively short half-life and relatively severe side effects, limiting its use. the delivery devices described herein may provide continuous delivery and may dramatically reduce side effects associated with both apomorphine and dopamine fluctuation. In some embodiments, a delivery device as described herein can provide continuous delivery of apomorphine or other dopamine agonist, with, optionally, an adjustable baseline and/or a bolus button for treating and “off” state in the patient. Advantageously, this method of treatment may provide improved dopaminergic levels in the body, such as fewer dyskinetic events, fewer “off” states, less total time in “off” states, less cycling between “on” and “off” states, and reduced need for levodopa; quick recovery from “off” state if it occurs; and reduced or eliminated nausea/vomiting side effect of apomorphine, resulting from slow steady infusion rather than bolus dosing.

In some embodiments, a delivery device as described herein may be used for administration of an analgesic, such as morphine, hydromorphone, fentanyl or other opioids, in the treatment of pain. Advantageously, the delivery device may provide improved comfort in a less cumbersome and/or less invasive technique, such as for post-operative pain management. Particularly, the delivery device may be configured for patient-controlled analgesia.

### CONCLUSION

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

For example, although various embodiments have been described as having particular features and/or combinations of components, other embodiments are possible having any combination or sub-combination of any features and/or components from any of the embodiments described herein. The specific configurations of the various components can also be varied. For example, the size and specific shape of the various components can be different than the embodiments shown, while still providing the functions as described herein. In addition, although the adaptor plate (118, 518, 618, 718) was described herein with reference to use with particular embodiments of a drug delivery device, an adaptor plate can also be included in other embodiments of a drug delivery device to nest or stack multiple electrochemical actuators. In another example, the cover 1070 can be configured to envelop and retain any combination of a first actuator (e.g., 102, 202, 302, 402, 502, 602, 702, 802, 902, 1002), an adaptor (e.g., 118, 218, 318, 418, 518, 618, 718, 818, 918, 1018), and a second actuator (e.g., 130, 230, 330, 430, 530, 630, 730, 830, 930, 1030). Specifically, for example, a cover can be used to couple the first actuator 802, the adaptor plate 818, and the second actuator 830. In still another example, an adaptor plate can have a combination of one or more recesses and an aperture, such as an aperture 846 extended between recesses 546, 552. In yet another example, although the adaptor 918 has been illustrated and described as including four clips 922, 924, 926, 928, in other embodiments, an adaptor can include any suitable number of clips, such as two, three, five, or more clips.

What is claimed is:

1. An apparatus, comprising:
a reservoir configured to contain a fluid;
a first actuator coupled to the reservoir;
a second actuator coupled to the first actuator, the first actuator and the second actuator collectively configured to exert a force on the reservoir such that at least a portion of the fluid within the reservoir is communicated out of the reservoir; and
an adaptor at least partially disposed between the first actuator and the second actuator, the adaptor configured to couple the first actuator and the second actuator.

2. The apparatus of claim 1, further comprising:
a transfer structure disposed between the first actuator and the reservoir, the transfer structure configured to contact the reservoir upon actuation of at least one of the first actuator or the second actuator.

3. The apparatus of claim 1, wherein the first actuator is an electrochemical actuator and the second actuator is an electrochemical actuator.

4. The apparatus of claim 1, wherein the adaptor includes an adaptor plate defining a first recess configured to receive a portion of the first actuator and a second recess configured to receive a portion of the second actuator.

5. The apparatus of claim 4, wherein the first recess defines a first longitudinal axis and the second recess defines a second longitudinal axis, the first longitudinal axis being transverse to the second longitudinal axis.

6. The apparatus of claim 4, wherein the first recess defines a first longitudinal axis and the second recess defines a second longitudinal axis, the first longitudinal axis being parallel to the second longitudinal axis.

7. The apparatus of claim 1, wherein the adaptor includes an adaptor plate defining an aperture in a medial portion of the adaptor plate, the medial portion being disposed between a first end of the adaptor plate and a second end of the adaptor plate.

8. The apparatus of claim 1, wherein the adaptor includes a first clip disposed about an end portion of the first actuator, the adaptor includes a second clip disposed about an end portion of the second actuator, the first clip configured to move relative to the second clip when at least one of the first actuator or the second actuator is actuated.

9. The apparatus of claim 1, wherein:
the first actuator has a first end, a second end, and a medial portion between its first end and its second end, the first actuator being configured so that when actuated, the first actuator bends in the medial portion in a first direction with respect to the adaptor; and
the second actuator has a first end, a second end, and a medial portion between its first end and its second end, the second actuator being configured so that when actuated, the second actuator bends in the medial portion in a second direction with respect to the adaptor, the second direction opposite the first direction.

10. The apparatus of claim 1, wherein the first actuator is configured to bend along a first plane during actuation, the adaptor is configured to substantially prevent bending of the first actuator along a second plane different than the first plane during actuation.

11. An apparatus, comprising:
a reservoir configured to contain a fluid;
a first actuator having a first configuration and a second configuration, the first actuator configured to exert a first force on the reservoir when the first actuator moves from its first configuration to its second configuration;
a second actuator having a first configuration and a second configuration, the second actuator configured to exert a second force on the reservoir when the second actuator moves from its first configuration to its second configuration;
an adaptor defining a first recess configured to receive a portion of the first actuator and a second recess configured to receive a portion of the second actuator,
the first actuator when moved from its first configuration to its second configuration defining a first stroke, the second actuator when moved from its first configuration to its second configuration defining a second stroke, a stroke of the apparatus being collectively defined by a sum of the first stroke and the second stroke.

12. The apparatus of claim 11, wherein at least one of the first actuator and the second actuator includes an electrochemical actuator.

13. The apparatus of claim 11, wherein the first actuator and the second actuator are configured to be actuated substantially simultaneously.

14. The apparatus of claim 11, wherein the first actuator and the second actuator are in a stacked configuration.

15. The apparatus of claim 11, further comprising:
a transfer structure disposed between at least one of the first actuator and the second actuator and the reservoir, the transfer structure configured to transfer the stroke of the apparatus onto the reservoir.

16. The apparatus of claim 11, wherein the adaptor is configured to couple the first actuator and the second actuator, at least a portion of the adaptor disposed between the first actuator and the second actuator.

17. The apparatus of claim 11, further comprising:
a cover substantially enveloping the first actuator and the second actuator, the cover configured to retain the first actuator with respect to the second actuator, the cover configured to permit deformation of the first actuator during actuation.

18. An apparatus, comprising:
a reservoir configured to contain a fluid;
a first actuator having a first configuration in which it is substantially planar and a second configuration in which at least a portion of the first actuator moves substantially perpendicular to the plane of its first configuration, the first actuator configured to exert a first force on the reservoir when the first actuator moves from its first configuration to its second configuration to urge fluid within the reservoir out of the fluid reservoir;
a second actuator having a first configuration and a second configuration, the second actuator configured to exert a second force on the reservoir when the second actuator moves from its first configuration to its second configuration to urge fluid within the reservoir out of the fluid reservoir; and
an adaptor at least partially disposed between the first actuator and the second actuator.

19. The apparatus of claim 18, wherein the second actuator is substantially planar when the second actuator is in its first configuration, the plane of the second actuator being substantially parallel with the plane of the first actuator, at least a portion of the second actuator moves substantially perpendicular to the plane of its first configuration when the second actuator moves to its second configuration.

20. The apparatus of claim 19, wherein the portion of the first actuator moves in a first direction when the first actuator moves to its second configuration, the portion of the second actuator moves in a second direction opposite the first direction when the second actuator moves to its second configuration.

21. The apparatus of claim 18, wherein the first volume of fluid and the second volume of fluid are communicated out of the fluid reservoir simultaneously.

22. The apparatus of claim 18, wherein the first volume of fluid and the second volume of fluid are communicated out of the fluid reservoir sequential.
